SEQUENCE LISTING (60 sequences)

SEQ ID NO: 1 - DNA, 30 bp, Artificial Sequence
  Description of Artificial Sequence Synthetic primer
atggacgcaactgtgaacatgatgttcgca

SEQ ID NO: 2 - DNA, 30 bp, Artificial Sequence
  Description of Artificial Sequence Synthetic primer
ctttgaggtcctggtccatcacgtgaccat

SEQ ID NO: 3 - DNA, 24 bp, Artificial Sequence
  Description of Artificial Sequence Synthetic primer
actgcccctcatcagactgctact

SEQ ID NO: 4 - DNA, 24 bp, Artificial Sequence
  Description of Artificial Sequence Synthetic primer
cactgccttgtactcgggtagctg

SEQ ID NO: 5 - DNA, 20 bp, Artificial Sequence
  Description of Artificial Sequence Synthetic primer
caggtgtttgagggtagctc

SEQ ID NO: 6 - DNA, 20 bp, Artificial Sequence
  Description of Artificial Sequence Synthetic primer
cggttcatcatggtacagtc

SEQ ID NO: 7 - DNA, 30 bp, Artificial Sequence
  Description of Artificial Sequence Synthetic primer
attcttcgttgtcaagccgccaaagtggag

SEQ ID NO: 8 - DNA, 30 bp, Artificial Sequence
  Description of Artificial Sequence Synthetic primer
agttgtttgctgcggagttgtcatctcgtc

SEQ ID NO: 9 - DNA, 23 bp, Artificial Sequence
  Description of Artificial Sequence Synthetic primer
tagagctagactccgggcgatga

SEQ ID NO: 10 - DNA, 23 bp, Artificial Sequence
  Description of Artificial Sequence Synthetic primer
ttgccttaaacaagaccacgaaa

SEQ ID NO: 11 - DNA, 23 bp, Artificial Sequence
  Description of Artificial Sequence Synthetic primer
gctatctactgtgtgtcccagtc

SEQ ID NO: 12 - DNA, 20 bp, Artificial Sequence
  Description of Artificial Sequence Synthetic primer
agagaaggatgtggttcgag

SEQ ID NO: 13 - DNA, 20 bp, Artificial Sequence
  Description of Artificial Sequence Synthetic primer
attcaggcgtggtagacatc

SEQ ID NO: 14 - DNA, 20 bp, Artificial Sequence
  Description of Artificial Sequence Synthetic primer
gtggggcgcaaaatgtctag

SEQ ID NO: 15 - DNA, 20 bp, Artificial Sequence
  Description of Artificial Sequence Synthetic primer
tctgagtacataagggccac

SEQ ID NO: 16 - DNA, 20 bp, Artificial Sequence
  Description of Artificial Sequence Synthetic primer
gtagactttcgtaagtcccc

SEQ ID NO: 17 - DNA, 20 bp, Artificial Sequence
  Description of Artificial Sequence Synthetic primer
tttccggaggaagcctgaac

SEQ ID NO: 18 - DNA, 20 bp, Artificial Sequence
  Description of Artificial Sequence Synthetic primer
ctcctgatcctcttatctgg

SEQ ID NO: 19 - DNA, 21 bp, Artificial Sequence
  Description of Artificial Sequence Synthetic primer
aagcgactcaccctggctgac

SEQ ID NO: 20 - DNA, 21 bp, Artificial Sequence
  Description of Artificial Sequence Synthetic primer
gactatgccatcactcgctgc

SEQ ID NO: 21 - DNA, 21 bp, Artificial Sequence
  Description of Artificial Sequence Synthetic primer
ggactacaatggccagatcaa

SEQ ID NO: 22 - DNA, 20 bp, Artificial Sequence
  Description of Artificial Sequence Synthetic primer
ggaaggcactgaaacaggac

SEQ ID NO: 23 - DNA, 20 bp, Artificial Sequence
  Description of Artificial Sequence Synthetic primer
aggaaagacctccagcgtgt

SEQ ID NO: 24 - DNA, 20 bp, Artificial Sequence
  Description of Artificial Sequence Synthetic primer
tctcttcggcttcaacgtct

SEQ ID NO: 25 - DNA, 20 bp, Artificial Sequence
  Description of Artificial Sequence Synthetic primer
tccagcacattgaagaggtg

SEQ ID NO: 26 - DNA, 20 bp, Artificial Sequence
  Description of Artificial Sequence Synthetic primer
gcgaggagaagtctgtgctc

SEQ ID NO: 27 - DNA, 20 bp, Artificial Sequence
  Description of Artificial Sequence Synthetic primer
ggagggaaaaccacaagaca

SEQ ID NO: 28 - DNA, 20 bp, Artificial Sequence
  Description of Artificial Sequence Synthetic primer
ctgtcttcaacctggcgtct

SEQ ID NO: 29 - DNA, 20 bp, Artificial Sequence
  Description of Artificial Sequence Synthetic primer
caacagtgggctcaacttca

SEQ ID NO: 30 - DNA, 20 bp, Artificial Sequence
  Description of Artificial Sequence Synthetic primer
gggggtaactgagcacaatc

SEQ ID NO: 31 - DNA, 20 bp, Artificial Sequence
  Description of Artificial Sequence Synthetic primer
aggacacgttcaagggaatg

SEQ ID NO: 32 - DNA, 20 bp, Artificial Sequence
  Description of Artificial Sequence Synthetic primer
actgtcaccccttcaaatgc

SEQ ID NO: 33 - DNA, 20 bp, Artificial Sequence
  Description of Artificial Sequence Synthetic primer
cgttcaccgagatctcaagc

SEQ ID NO: 34 - DNA, 20 bp, Artificial Sequence
  Description of Artificial Sequence Synthetic primer
gaacgtctggaggaacttgc

SEQ ID NO: 35 - DNA, 18 bp, Artificial Sequence
  Description of Artificial Sequence Synthetic primer
atctgcacagcggagagg

SEQ ID NO: 36 - DNA, 20 bp, Artificial Sequence
  Description of Artificial Sequence Synthetic primer
cgtgatttggttgatgttgc

SEQ ID NO: 37 - DNA, 20 bp, Artificial Sequence
  Description of Artificial Sequence Synthetic primer
cctgcagcaagtgcaactac

SEQ ID NO: 38 - DNA, 24 bp, Artificial Sequence
  Description of Artificial Sequence Synthetic primer
gcttgagtaaggacaaagttcaca

SEQ ID NO: 39 - DNA, 20 bp, Artificial Sequence
  Description of Artificial Sequence Synthetic primer
ccatgaacaaggaagggaaa

SEQ ID NO: 40 - DNA, 20 bp, Artificial Sequence
  Description of Artificial Sequence Synthetic primer
tccgctgtgtgtccatttag

SEQ ID NO: 41 - DNA, 20 bp, Artificial Sequence
  Description of Artificial Sequence Synthetic primer
cagtgacacaggcaggtcag

SEQ ID NO: 42 - DNA, 20 bp, Artificial Sequence
  Description of Artificial Sequence Synthetic primer
gggacagtgagagaggttgc

SEQ ID NO: 43 - DNA, 20 bp, Artificial Sequence
  Description of Artificial Sequence Synthetic primer
atgggattggatacgttgga

SEQ ID NO: 44 - DNA, 20 bp, Artificial Sequence
  Description of Artificial Sequence Synthetic primer
ccggctgacataagctcttt

SEQ ID NO: 45 - DNA, 20 bp, Artificial Sequence
  Description of Artificial Sequence Synthetic primer
ctgaggtttccgtgtccagt

SEQ ID NO: 46 - DNA, 20 bp, Artificial Sequence
  Description of Artificial Sequence Synthetic primer
ttctgccactctttgcagtg

SEQ ID NO: 47 - DNA, 20 bp, Artificial Sequence
  Description of Artificial Sequence Synthetic primer
ttcaccaccatggagaaggc

SEQ ID NO: 48 - DNA, 18 bp, Artificial Sequence
  Description of Artificial Sequence Synthetic primer
cccttttggctccaccct

SEQ ID NO: 49 - DNA, 24 bp, Artificial Sequence
  Description of Artificial Sequence Synthetic primer
cccatggtggtggtacgggaattc

SEQ ID NO: 50 - DNA, 20 bp, Artificial Sequence
  Description of Artificial Sequence Synthetic primer
tctcggtctcggacaaaagt

SEQ ID NO: 51 - DNA, 24 bp, Artificial Sequence
  Description of Artificial Sequence Synthetic primer
cccatggtggtggtacgggaattc

SEQ ID NO: 52 - DNA, 20 bp, Artificial Sequence
  Description of Artificial Sequence Synthetic primer
gtcgttgaactcctcggtct

SEQ ID NO: 53 - DNA, 24 bp, Artificial Sequence
  Description of Artificial Sequence Synthetic primer
cccatggtggtggtacgggaattc

SEQ ID NO: 54 - DNA, 20 bp, Artificial Sequence
  Description of Artificial Sequence Synthetic primer
agttgctttccactcgtgct

SEQ ID NO: 55 - DNA, 20 bp, Artificial Sequence
  Description of Artificial Sequence Synthetic primer
ctcctggcaaaaggtcagag

SEQ ID NO: 56 - DNA, 20 bp, Artificial Sequence
  Description of Artificial Sequence Synthetic primer
tcggttgttgctgatctgtc

SEQ ID NO: 57 - DNA, 20 bp, Artificial Sequence
  Description of Artificial Sequence Synthetic primer
tgttaccaactgggacgaca

SEQ ID NO: 58 - DNA, 20 bp, Artificial Sequence
  Description of Artificial Sequence Synthetic primer
tctcagctgtggtggtgaag

SEQ ID NO: 59 - DNA, 20 bp, Artificial Sequence
  Description of Artificial Sequence Synthetic primer
atccacgctgttttgacctc

SEQ ID NO: 60 - DNA, 20 bp, Artificial Sequence
  Description of Artificial Sequence Synthetic primer
cgaagtctgaagccaggtgt

(19) United States
(12) Patent Application Publication
(10) Pub. No.: US 2010/0184051 A1
(43) Pub. Date: Jul. 22, 2010

(54) METHODS OF GENERATING PLURIPOTENT CELLS FROM SOMATIC CELLS

(75) Inventors: Konrad Hochedlinger, Boston, MA (US); Nimet Maherali, Boston, MA (US)

Correspondence Address: DAVID S. RESNICK, NIXON PEABODY LLP, 100 SUMMER STREET, BOSTON, MA, 02110-2131, US

(73) Assignees: THE GENERAL HOSPITAL CORPORATION, Boston, MA (US); PRESIDENT AND FELLOWS OF HARVARD COLLEGE, Cambridge, MA (US)

(21) Appl. No.: 12/602,184

(22) PCT Filed: May 30, 2008
(86) PCT No.: PCT/US08/65384

§ 371 Date: Nov. 30, 2009

Related U.S. Application Data
(60) Provisional application No. 60/932,267, filed on May 30, 2007.

(51) Int. Cl. C12Q 1/68

(52) U.S. Cl. 435/6

(57) ABSTRACT

Disclosed herein are methods to select for the generation of mouse and human pluripotent stem cells during developmental reprogramming. The methods described herein relate to the selection of induced pluripotent stem cells, i.e., pluripotent stem cells generated or induced from differentiated cells without a requirement for genetic selection. Described herein are particular embodiments for selection of reprogrammed cells based on 1) colony morphology, or 2) X chromosome reactivation in female cells.

This application claims priority under 35 U.S.C. §119(e) to U.S. Provisional Application Ser. No. 60/932,267, filed May 30, 2007, the entirety of which is incorporated herein by reference.

### BACKGROUND OF THE INVENTION

Reprogramming of cells by nuclear transfer (Wakayama, T., Perry, A. C., Zuccotti, M., Johnson, K. R., and Yanagimachi, R. (1998) Nature 394, 369-374; Wilmut, I., Schnieke, A. E., McWhir, J., Kind, A. J., and Campbell, K. H. (1997) Nature 385, 810-813) and cell fusion (Cowan, C. A., Atienza, J., Melton, D. A., and Eggan, K. (2005) Science 309, 1369-1373; Tada, M., Takahama, Y., Abe, K., Nakatsuji, N., and Tada, T. (2001) Curr Biol 11, 1553-1558) allows for the re-establishment of a pluripotent state in a somatic nucleus (Hochedlinger, K., and Jaenisch, R. (2006) Nature 441, 1061-1067). While the molecular mechanisms of nuclear reprogramming are not fully elucidated, cell fusion experiments have implied that reprogramming factors can be identified in ES cells and be used to directly induce reprogramming in somatic cells. Indeed, a rational approach recently led to the identification of four transcription factors whose expression enabled the induction of a pluripotent state in adult fibroblasts (Takahashi, K., and Yamanaka, S. (2006) Cell 126, 663-676). Yamanaka and colleagues demonstrated that retroviral expression of the transcription factors Oct4, Sox2, c-Myc, and Klf4, combined with genetic selection for Fbx15 expression, gives rise to iPS cells directly from fibroblast cultures. Fbx15-selected iPS cells contributed to diverse tissues in mid-gestation embryos, however, these embryos succumbed at midgestation, indicating a restricted developmental potential of iPS cells compared with ES cells. Consistent with this observation, only part of the ES cell transcriptome was expressed in iPS cells, and methylation analyses of the chromatin state of the Oct4 and Nanog promoters demonstrated an epigenetic pattern that was intermediate between that of fibroblasts and ES cells.

These observations raised three fundamental questions about the molecular and functional nature of directly reprogrammed cells: (i) can selection for a gene that is essential for the ES cell state generate pluripotent cells that are more similar to ES cells than the previously described Fbx15-selected iPS cells; (ii) does the pluripotent state of iPS cells depend on continuous expression of exogenous factors; and (iii) does transcription factor-induced reprogramming reset the epigenetic landscape of a fibroblast genome into that of a pluripotent cell.

Successful reprogramming of somatic cells by nuclear transfer or cell fusion is thought to require faithful remodeling of epigenetic modifications such as DNA methylation, histone modifications, and reactivation of a silent X chromosome in female cells (Rideout, W. M., 3rd, Eggan, K., and Jaenisch, R. (2001) Science 293, 1093-1098). Aberrant epigenetic reprogramming is assumed to be the principal reason for the developmental failure and abnormalities seen in animals cloned by nuclear transfer. Thus, the question of epigenetic reprogramming is of particular relevance for the potential therapeutic applications of iPS cells, as epigenetic aberrations can result in pathological conditions such as cancer (Gaudet, F., Hodgson, J. G., Eden, A., Jackson-Grusby, L., Dausman, S., Gray, J. W., Leonhardt, H., and Jaenisch, R. (2003) Science 300, 489-492).

### SUMMARY OF THE INVENTION

The methods described herein relate to the selection of induced pluripotent stem cells—that is, pluripotent stem cells generated or induced from differentiated cells, including, for example, adult fibroblasts. The induction of pluripotency by inducing the expression of a limited number of transcription factors has been demonstrated in the art and can be applied to any mammalian cell, non-human mammalian cell or human cell.

Methods described herein permit selection for the generation of mammalian (including for example, mouse and human) pluripotent cells during developmental reprogramming. The over-expression of a defined set of transcription factors can convert adult somatic cells into embryonic stem (ES) cell-like cells, however, this process generally requires genetic selection for the reactivation of ES cell-specific genes; the absence of selection results in the generation of many non-ES-like cells in addition to the ES-like cells. Such genetic selection techniques are generally not feasible in human cells and are generally nor desirable for cells to be introduced to a human patient. To address this issue, described herein are novel selection strategies that permit one to select for reprogrammed cells based on 1) colony morphology only, and 2) X chromosome reactivation in female cells. That is, in the absence of genetic selection, chemical selection, or both.

Morphology-based selection requires a much longer time period for reprogramming relative to existing selection approaches, on the order of one to two months following the addition of reprogramming factors. After this time, ES-like colonies can be picked and expanded. Many non-ES-like cells remain at the time picking but, upon passaging the cells e.g., at clonal density, ES-like colonies can readily be recovered and cell lines can be generated.

Selection based on X chromosome reactivation takes advantage of female cell lines that are heterozygous for mutations in the Hprt locus. It is shown herein that X chromosome reactivation occurs during reprogramming by defined factors, and this event occurs late in the reprogramming process (on the order of 3-4 weeks). In female somatic cells, only one X chromosome is active, while the other is silent. In one aspect, in Hprt heterozygous cells, those that harbor a mutant Hprt gene on the active X chromosome will be resistant to 6-thioguanine. Upon reprogramming and X chromosome reactivation, these cells express the normal Hprt gene and gain resistance to HAT medium, while losing resistance to 6-thioguanine.

One aspect of the methods described herein permits the selection of induced pluripotent stem cells, comprising the steps of: a) re-programming a differentiated primary cell to a pluripotent phenotype, wherein the differentiated primary cell does not express Nanog mRNA when measured by RT-PCR; b) culturing the cell re-programmed in step (a) in the absence of a selection agent after re-programming; c) microscopically observing the culture of step (b), and isolating a clone of cells in the culture which have become smooth and rounded in appearance; and d) testing cells of the clone for the expression of a stem cell marker; wherein the detection of stem cell marker expression is indicative that the cells are induced pluripotent stem cells.

In one embodiment of this aspect and all other aspects described herein, the re-programming comprises one of: introducing nucleic acid sequences encoding the transcription factors Oct4, Sox2, c-Myc and Klf4 to the differentiated somatic cell, the sequences operably linked to regulatory elements for the expression of the factors; introducing one or more protein factors that re-program the cell's differentiation state; and contacting the cell with a small molecule that induces a re-programming of the cell's differentiated state.

In another embodiment of this aspect and all other aspects described herein, the method further comprises the step of introducing cells of a clone that express a stem cell marker into nude mice and performing histology on a tumor arising from the cells, wherein the growth of a tumor comprising cells from all three germ layers further indicates that the cells are pluripotent stem cells.

In another embodiment of this aspect and all other aspects described herein, the step of culturing further comprises passaging the cells.

In another embodiment of this aspect and all other aspects described herein, the differentiated somatic cell has a morphology distinctly different from that of an ES cell.

In another embodiment of this aspect and all other aspects described herein, the differentiated primary cell is a fibroblast, and wherein the fibroblast is flattened and irregularly shaped prior to re-programming.

In another embodiment of this aspect and all other aspects described herein, the stem cell marker is selected from the group consisting of SSEA1, CD9, Nanog, Fbx15, Ecat1, Esg1, Eras, Gdf3, Fgf4, Cripto, Dax1, Zpf296, Slc2a3, Rex1, Utf1, and Oct4.

In another embodiment of this aspect and all other aspects described herein, the method further comprises the step of testing cells of the clone for the reactivation of an inactive X chromosome, when the differentiated primary cell is from a female individual.

In another embodiment of this aspect and all other aspects described herein, the nucleic acid sequences are comprised in a viral vector or a plasmid.

In another embodiment of this aspect and all other aspects described herein, the viral vector is a retroviral vector, a lentiviral vector or an adenoviral vector.

In another embodiment of this aspect and all other aspects described herein, the method further comprises the step of testing cells of the clone for the expression of exogenous Oct4, Sox2, c-Myc and/or Klf4.

In another embodiment of this aspect and all other aspects described herein, the primary cell comprises a human cell.

Another aspect described herein is a method of selecting induced pluripotent stem cells, the method comprising: a) providing a female cell that is heterozygous for a selectable marker on the X chromosome, wherein the selectable marker is mutant on the active X chromosome and wild-type on the inactive X chromosome, and wherein the cell does not express Nanog mRNA when measured by RT-PCR; b) re-programming the cell to a pluripotent phenotype; and c) culturing the cell with a selection agent, wherein the reactivation of the inactive X chromosome permits the expression of wild-type selectable marker and permits cell survival in the presence of the selection agent, whereby surviving cells are induced pluripotent stem cells.

In one embodiment of this aspect, the method further comprises the step of testing a cell surviving in the presence of the selection agent for the expression of a stem cell marker.

In another embodiment of this aspect and all other aspects described herein, the stem cell marker is selected from the group consisting of SSEA1, CD9, Nanog, Fbx15, Ecat1, Esg1, Eras, Gdf3, Fgf4, Cripto, Dax1, Zpf296, Slc2a3, Rex1, Utf1, and Oct4.

In another embodiment of this aspect and all other aspects described herein, the re-programming comprises one of: introducing nucleic acid sequences encoding the transcription factors Oct4, Sox2, c-Myc and Klf4 to the differentiated somatic cell, the sequences operably linked to regulatory elements for the expression of the factors; introducing one or more protein factors that re-program the cell's differentiation state; and contacting the cell with a small molecule that induces a re-programming of the cell's differentiated state.

In another embodiment of this aspect and all other aspects described herein, the method further comprises the step of introducing cells that survive in the presence of the selection agent into nude mice and performing histology on a tumor arising from the cells, wherein the growth of a tumor comprising cells from all three germ layers further indicates that the cells are pluripotent stem cells.

In another embodiment of this aspect and all other aspects described herein, the cell is a cell of a cell line.

In another embodiment of this aspect and all other aspects described herein, the cell is heterozygous for a mutant Hprt gene on the X chromosome.

In another embodiment of this aspect and all other aspects described herein, the cell carries a wild-type Hprt gene on the X chromosome that is inactive before the introduction of the nucleic acids and a mutant, non-functional Hprt gene on the X chromosome that is active before re-programming.

In another embodiment of this aspect and all other aspects described herein, the cell is resistant to 6-thioguanine before re-programming.

In another embodiment of this aspect and all other aspects described herein, the selection agent comprises HAT medium.

In another embodiment of this aspect and all other aspects described herein, the cell comprises a human cell.

Another aspect described herein is a method of selecting induced pluripotent stem cells, the method comprising: a) providing a female cell which carries an X-chromosome-linked reporter gene that is subject to silencing by X inactivation, and wherein the female cell does not express Nanog mRNA when measured by RT-PCR; b) re-programming the cell to a pluripotent phenotype; c) culturing the cell after re-programming; and d) isolating a clone of cells from the culture which expresses the X-chromosome-linked reporter; wherein the expression of the reporter is indicative that the clone comprises induced pluripotent stem cells.

In one embodiment of this aspect and all other aspects described herein, the method further comprises the step of testing cells of the clone for the expression of a stem cell marker.

In another embodiment of this aspect and all other aspects described herein, the stem cell marker is selected from the group consisting of SSEA1, CD9, Nanog, Fbx15, Ecat1, Esg1, Eras, Gdf3, Fgf4, Cripto, Dax1, Zpf296, Slc2a3, Rex1, Utf1, and Oct4.

In another embodiment of this aspect and all other aspects described herein, the method further comprises the step of introducing cells that express the reporter into nude mice and performing histology on a tumor arising from the cells, wherein the growth of a tumor comprising cells from all three germ layers further indicates that the cells are pluripotent stem cells.

In another embodiment of this aspect and all other aspects described herein, the cell comprises a human cell.

### DEFINITIONS

The term “pluripotent” as used herein refers to a cell with the capacity, under different conditions, to differentiate to more than one differentiated cell type, and preferably to differentiate to cell types characteristic of all three germ cell layers. Pluripotent cells are characterized primarily by the ability to differentiate to more than one cell type, preferably to all three germ layers, using, for example, a nude mouse teratoma formation assay (see Examples herein). Pluripotency is also evidenced by the expression of embryonic stem (ES) cell markers, although the preferred test for pluripotency is the demonstration of the capacity to differentiate into cells of each of the three germ layers.

The term “re-programming” as used herein refers to the process of altering the differentiated state of a terminally-differentiated somatic cell to a pluripotent phenotype.

By “differentiated primary cell” is meant any primary cell that is not, in its native form, pluripotent as that term is defined herein. It should be noted that placing many primary cells in culture can lead to some loss of fully differentiated characteristics. However, simply culturing such cells does not, on its own, render them pluripotent. The transition to pluripotency requires a re-programming stimulus beyond the stimuli that lead to partial loss of differentiated character in culture. Re-programmed pluripotent cells also have the characteristic of the capacity of extended passaging without loss of growth potential, relative to primary cell parents, which generally have capacity for only a limited number of divisions in culture.

The term “vector” refers to a small carrier DNA molecule into which a DNA sequence can be inserted for introduction into a host cell where it will be replicated. An “expression vector” is a specialized vector that contains a gene with the necessary regulatory regions needed for expression in a host cell. The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

### BRIEF DESCRIPTION OF THE FIGURES

FIG. 1: ES cell-like properties of Nanog-selected IFS cells

(A) RT-PCR analysis of ES cell marker gene expression in Nanog-GFP (NGiP) ES cells, and two iPS cell lines grown with and without continued puromycin selection, as well as in wildtype ES cells (V6.5) and MEFs as additional reference points. Primers for Oct4 and Sox2 are specific for transcripts from the respective endogenous locus. Nat1 was used as a loading control.

(B) Western blot analysis for expression of Nanog, Oct4, Sox2, c-myc, and Klf4 in iPS cell lines, MEFs and NGiP-ES cells. Anti-tubulin and anti-actin antibodies were used to control for loading.

(C) Quantitative PCR analysis of pMX retroviral transcription in 1) wild-type MEFs, 2) wild-type ES cells, 3) cells from the heterogeneous iPS line 1A2 before sorting and subcloning, 4) 1D4 iPS, 5) 2D4 iPS, and 6) MEFs infected with the respective pMX virus. Transcript levels were normalized to β-Actin. It should be noted that the retroviruses in the 2D4 iPS line appear completely silenced while the heterogeneous 1A2 line still shows abundant expression of the exogenous factors.

FIG. 2: Fusion of IFS cells with somatic cells

(A) Schematic of cell fusion between 2D4 iPS cells and hygromycin resistant MEFs that carry an Oct4Neo selectable allele.

(B) DNA content analysis of 2D4 iPS cells, MEFs, and 2D4/MEF cell hybrids maintained either under puromycin/hygromycin selection or puromycin/G418 selection.

FIG. 3: Requirement for exogenous Oct4 for the maintenance of iPS cells

(A) Schematic of iPS cell generation using Oct4-inducible fibroblasts.

(B) MEFs infected with Sox2, c-MYC, and Klf4, in the absence or presence of doxycycline-inducible Oct4 expression. Shown are plates stained for alkaline phosphatase.

(C) Quantitative PCR analysis of Oct4 levels in Oct4-inducible iPS cells. Levels of transcripts from the endogenous and inducible allele were measured in undifferentiated iPS cells (+LIF, −dox), differentiated iPS cells (−LIF, −dox), and differentiated iPS cells re-induced at 5 days after LW withdrawal (−LW, +dox). Transcript levels were normalized to 13-Actin. ES cells carrying the inducible Oct4 allele; and wild-type MEFs served as controls.

FIG. 4: Gene-specific and global DNA methylation status in iPS cells

(A) Bisulfite sequencing of the Oct4 and Nanog promoter regions in ES cells, 2D4 iPS cells, and MEFs. Promoter regions containing the differentially methylated CpGs are shown with respect to the transcriptional start site (arrow). Open circles represent unmethylated CpGs; closed circles denote methylated CpGs.

(B) Bisulfite sequencing of the Nanog promoter in cell hybrids generated through fusion of iPS 2D4 cells and MEFs. Data shown for puromycin/hygromycin resistant hybrids as in FIG. 2C.

(C) Southern blot analysis of global DNA methylation using a satellite repeat probe. Genomic DNA from MEFs, male Nanog-GFP ES cells, female ES cells, iPS 2D4 parental cells and three subclones, was digested with the methylation-sensitive restriction enzyme HpaII and hybridized with a minor satellite repeat probe. Male ES cell DNA digested with the non-methylation sensitive isoschizomer MspI served as a control. Lower molecular weight bands are indicative of hypomethylation.

FIG. 5: X chromosome dynamics in IFS cells

(A) RT-PCR analysis of Xite intergenic transcripts in iPS cell line 2D4, NGiP MEFs, and male control ES cells. Transcripts at different locations along the Xite locus were detected (regions 5-7). Positive control, Rrm2, a house keeping gene. Like female ES cells, male ES cells express Xite transcripts.

(B) Enrichment of Ezh2 and H3me3K27 on the Xi in differentiating 2D4 iPS. The graphs show the percentage of cells with Xist RNA coating that show co-localization with Ezh2 or H3me3K27 on the Xi at different time points during retinoic acid-induced differentiation of 2D4 iPS cells (n>100 for each time point).

FIG. 6: Random X-inactivation in differentiating TTF-derived iPS cells

(A) Flow scheme for obtaining iPS cells from X^{GFP}X TTFs and for subsequent analysis of X-inactivation. X^{GFP}X TTFs carrying the Oct4-Neo allele were sorted at two consecutive passages to obtain a GFP negative population (Xi^{GFP}Xa; <0.05% green cells). Reprogrammed cells were selected based on ES cell morphology and GFP reactivation. Drug selection with G418 was employed to retrospectively verify the reprogrammed state of the iPS cells but not to select for iPS cell establishment. iPS cells were subcloned, differentiated, and analyzed by FACS and Xist FISH. Numbers of GFP+ or GFP− cells determined by FACS are given in orange, while the numbers given in blue indicate the percentage of cells with Xist RNA coating of the Xi within GFP+ and GFP− differentiated iPS cells, respectively.

FIG. 7: Global analysis of H3K4 and H3K27 trimethylation in iPS cells

(A) Global correlation of K4 and K27 trimethylation data between all cell types. The table shows the binary global correlation of K4 and K27 trimethylation, respectively, between all possible pairs of cell types and for all genes on the array (˜16500).

(B) Correlation of K4 and K27 trimethylation within E class genes between all cell types. Correlation values for K4 or K27 methylation for each two pairs of cell types were plotted as a function of the distance from the transcription start site in increments of 500 bp.

FIG. 8: In vivo developmental potential of Nanog-selectable iPS cells

(A) Cells from iPS line 2D4 that carried a randomly integrated GFP transgene were injected into blastocysts. Surrogate mothers gave birth to GFP-positive pups. A non-chimeric pup not expressing GFP is shown.

(B) Flow cytometric analysis of hematopoietic cells isolated from the spleen and thymus of a newborn iPS cell derived chimeric mouse. Histograms denote the percentage of GFP-positive cells in populations gated on lineage-specific markers.

(C) 10-day old chimeric mouse derived from blastocyst-injected 2D4 iPS cells, shown next to a wild-type littermate (iPS-derived cells are responsible for the agouti coat color).

FIG. 9: Analysis of retroviral integration DNA imprint status in iPS cell lines 1A2 and 2D4

(A) Analysis of retroviral integration sites in iPS cells.

Retroviral integrations were determined by Southern blot analysis. DNA was digested with BamHI (for Oct4, and Klf4) or HindIII (for Sox2) or BglII (for c-MYC) and hybridized with the respective cDNA probes. Integrations are shown for V6.5 ES cells (wt) and the two iPS lines 1A2 and 2D4.

(B) Schematic drawing of the individual viral constructs including internal restriction sites used for integration site analysis. It should be noted that the cDNA probe will detect a restriction fragment generated by one pMX internal cut and one external cut in the genomic region in to which the virus has integrated.

(C) Methylation status at the Igf2r differentially methylated region. DNA from different cell types was digested using the PvuII and MIUI restriction enzymes and analyzed by Southern blotting. The methylated (M) and un-methylated (U) alleles are indicated. Only the un-methylated allele was detected in ES cells lacking Dnmt1 or in embryonic germ (EG) cells derived from the E12.5 embryos. The fact that imprinting is maintained in the iPS cells suggests that iPS cells are not derived from rare germ cells that may have contaminated the fibroblast culture.

FIG. 10: Statistical significance of signature gene analysis

The classification of most signature genes in 2D4 iPS cells as ES-like (E class) based on their methylation pattern is highly significant. The top panel shows the observed distribution of the 2D4 loci into E, N, and M classes (from data presented in FIG. 7A). To validate the classification of 2D4 loci into E, M, and N genes, 2D4 methylation data were permutated 100 times, randomly assigned to ES-MEF pairs, and signature genes re-classified at different stringencies (p=0.01, p=0.05, p=0.1) (bottom panel).

FIG. 11: Expression of signature genes in iPS cells

(A) Global correlation of the entire expression data sets (from Agilent microarrays) between V6.5 ES cells (ES), puromycin-selected Nanog GFP ires Puro ES cells (ESₚᵤᵣₒ), Nanog GFP ires Puro MEFs (MEF), and 2D4 iPS cells (iPS) determined by Pearson Correlation.

(B) Number of genes in the complete expression data sets of ESₚᵤᵣₒ, MEF, and iPS described in (A), which showed a more than 2 fold change in expression relative to ES cells.

(C) Real-time PCR analysis of transcript levels of 13 selected signature genes in 2D4 iPS cells, female MEFs, and V6.5 ES cells. To determine relative expression levels, RNA was prepared using the Qiagen RNA easy kit and 1 ug was reverse transcribed using the Omniscript RT kit (Qiagen) and random primers. Transcript levels were quantified by real time PCR and normalized to a Gapdh control using the ΔΔCt method. Expression in ES cells is set arbitrarily at 1 and error bars represent the standard deviation of triplicate reactions. Primer sequences are given in Table 3. Note the different scales of the Y-axis.

In agreement with our genome-wide expression data, two out of three tested genes belonging to the M class (Vg114, HoxD10) demonstrated an ES-like expression pattern in 2D4 iPS cells (lower expression in ES and iPS cells than in MEFs) even though they were classified as MEF-like genes based on their histone modification pattern. To this end, closer manual inspection of the histone methylation at these loci revealed that the repression seen in 2D4 iPS cells relative to MEFs correlates with a reduction in K4 methylation at these promoters in 2D4 iPS cells. All other genes showed a good correlation between K4 methylation only and relative higher expression, K27 methylation only and relative lower expression, and bivalency of histone H3 K4 and K27 methylation and lower expression.

FIG. 12: In vitro differentiation of iPS cells into hematopoietic lineages.

(A, B) Day 7 embryoid bodies derived from iPS cell line 2D4 and wildtype V6.5 ES cells were analyzed by flow cytometry for hematopoietic markers CD41 and c-kit marking immature hematopoietic cells (A), as well as CD45 and c-kit marking mature hematopoietic cells (B). The percentage of double positive cells is given. Note that in generating the EBs, a greater number of input cells were used for the iPS line than the V6.5 ES cell line, which may explain the quantitative differences in the percentage of differentiated cells.

(C) Mature hematopoietic cells obtained from a methylcellulose culture of dissociated day 7 EBs made from iPS cells. Multiple types of hematopoietic cells were present, including myeloblasts (i), macrophages (ii), mast cells (iii, iv), and early red blood cells (v,vi).

For the generation of blood cells, EBs were generated using the hanging drop method after elimination of the feeder cells by pre-plating (Geijsen, N., Horoschak, M., Kim, K., Gribnau, J., Eggan, K., and Daley, G. Q. (2004) Nature 427, 148-154). After three days, EBs were plated, and at day 7 EBs were dissociated into single cell suspensions with Collagenase IV for FACS analysis of hematopoietic markers (with antibodies described in Supplementary table 3) or for further in vitro differentiation. For methylcellulose cultures, a single cell suspension of day 7 EBs was mixed with methylcellulose supplemented with hematopoietic growth factors (M3434, Stem Cell Technologies) and seeded at 1×10⁵ cells per culture. After 10 days in culture, representative hematopoietic colonies were picked to prepare cytospins, which were counterstained with May-Gruenwald Giemsa.

### DETAILED DESCRIPTION

### Isolation of Induced Pluripotent Stem Cells in the Absence of Selection Agents

In one aspect, the methods described herein relate to the selection of induced pluripotent stem cells, which does not rely upon the use of selective agent(s) to identify or enrich for those cells that have become pluripotent, the methods relying instead upon changes in the morphology of the original cells occurring when cells take on the less differentiated, ES-like pluripotent phenotype.

In this aspect, the invention relates to a method of selecting induced pluripotent stem cells, the method having steps as follows. The first step involves the re-programming of a differentiated primary cell to a less differentiated or pluripotent state. Re-programming can be accomplished, for example, by transfer of the nucleus of a cell to an oocyte (see, e.g., Wilmut et al., 1997, Nature 385: 810-813), or by fusion with an existing embryonic stem cell (see, e.g., Cowan et al., 2005, Science 309: 1369-1373, and Tada et al., 2001, Curr. Biol. 11: 1553-1558). Such re-programming can also be done, for example, by introducing nucleic acid sequences encoding the transcription factors Oct4, Sox2, c-Myc and Klf4 to, for example, a fibroblast, the sequences operably linked to regulatory elements for the expression of the factors. While these factors are preferred, other transcription factors or a subset of these factors can also be employed (see, e.g., Takahashi & Yamanaka, 2006, Cell 126: 663-676, which is incorporated herein by reference).

In one embodiment, the transcription factors are encoded by a viral vector or a plasmid. The viral vector can be, for example, a retroviral vector, a lentiviral vector or an adenoviral vector. Non-viral approaches to the introduction of nucleic acids known to those skilled in the art can also be used with the methods described herein.

Alternatively, activation of the endogenous genes encoding such transcription factors can be used.

In another alternative, one or more protein factors that re-program the cell's differentiation state can be introduced to the cell. For example, protein factors (e.g., c-Myc, Oct4, Sox2 and/or Klf4, among others) can be introduced to the cell through the use of HIV-TAT fusion. The TAT polypeptide has characteristics that permit it to penetrate the cell, and has been used to introduce exogenous factors to cells (see, e.g., Peitz et al., 2002, Proc. Natl. Acad. Sci. USA. 99:4489-94). This approach can be employed to introduce factors for re-programming the cell's differentiation state. Finally, re-programming can be accomplished by contacting the cell with a small molecule that induces a re-programming of the cell' s differentiated state (see, e.g., Sato et al., 2004, Nature Med. 10:55-63).

While fibroblasts are preferred, other primary cell types can also be used. It is preferred that the parental cell have a morphology that is distinctly different from an ES cell, to facilitate the selection based on morphological change. By “distinctly different” is meant, at a minimum, that for adherent cells, the shape of the parental cell will be irregular, rather than rounded when grown in culture. For non-adherent primary cells, one can select first for adherence and then the rounded ES morphology. One of skill in the art knows the morphological characteristics of an ES cell, which tend to be rounded, rather than flat, and smooth, rather than rough, when viewed under phase contrast microscopy.

Further, the parental cell can be from any mammalian species, with non-limiting examples including a murine, bovine, simian, porcine, equine, ovine, or human cell. The parental cell should not express ES cell markers, e.g., Nanog mRNA or other ES markers. For clarity and simplicity, the description of the methods herein refers to fibroblasts as the parental cells, but it should be understood that all of the methods described herein can be readily applied to other primary parent cell types.

Where a fibroblast is used, the fibroblast is flattened and irregularly shaped prior to the re-programming, and does not express Nanog mRNA. The starting fibroblast will preferably not express other embryonic stem cell markers. The expression of ES-cell markers can be measured, for example, by RT-PCR. Alternatively, measurement can be by, for example, immunofluorescence or other immunological detection approach that detects the presence of polypeptides that are characteristic of the ES phenotype.

In the next step, following the introduction of nucleic acid sequences, the fibroblast is cultured in the absence of a selection agent. The term “in the absence of a selection agent” refers to the absence of a selection agent that selects for the induced pluripotent stem cell phenotype, e.g., the absence of a selection agent that selects for cells which have de-differentiated to express one or more ES cell markers. While it is preferred that there be no selection agents of any kind present, selection agents for the presence of the nucleic acids encoding the transcription factors Oct4, Sox2, c-Myc and Klf4 can be present, although the continued expression of these factors is not absolutely required for maintenance of the pluripotent phenotype (see below). The method can include testing for the presence or expression of the introduced transcription factors in an isolated clone.

In the next step, cells that are being cultured in the absence of a selection agent are microscopically observed (e.g., under ordinary phase contrast light microscopy or other appropriate optics) to identify cells in the cultures which have lost the irregular morphology characteristic of the parental cells, e.g., the flattened, irregular morphology of fibroblasts, and have become smooth and rounded in appearance. The cells round up but remain viable as they undergo the transition to pluripotency. The cells can be passaged to facilitate selection by morphology. Clones of viable cells that exhibit a rounded morphology are isolated, e.g., by limiting dilution and culture in multi-well plates or other approaches known to those of skill in the art.

In a further step, the isolated clones are tested for the expression of a stem cell marker. Such expression identifies the cells as induced pluripotent stem cells. Stem cell markers can be selected from the non-limiting group including SSEA1, CD9, Nanog, Fbx15, Ecat1, Esg1, Eras, Gdf3, Fgf4, Cripto, Dax1, Zpf296, Slc2a3, Rex1, Utf1, and Nat1. Methods for detecting the expression of such markers can include, for example, RT-PCR and immunological methods that detect the presence of the encoded polypeptides.

The pluripotent stem cell character of the isolated cells can be confirmed by any of a number of tests evaluating the expression of ES markers and the ability to differentiate to cells of each of the three germ layers. As one example, teratoma formation in nude mice can be used to evaluate the pluripotent character of the isolated clones. The cells are introduced to nude mice and histology is performed on a tumor arising from the cells. The growth of a tumor comprising cells from all three germ layers further indicates that the cells are pluripotent stem cells.

In another embodiment, where the cells are female, the re-activation of the inactive X chromosome can be evaluated as a measure of de-differentiation and pluripotency.

Selection by Monitoring X-Reactivation:

Inactivation of one of the X chromosomes in females is a hallmark of differentiation away from pluripotency. When cells are induced to the pluripotent state, e.g., by the expression of Oct4, Sox2, c-Myc and Klf4, the inactive X chromosome is re-activated.

Another aspect of the methods described herein uses the re-activation of an inactive X chromosome of differentiated female cells to select for induced pluripotent stem cells.

In this aspect, a method is provided for selecting induced pluripotent stem cells, the method having steps as follows. First, a female cell is provided that is heterozygous for a selectable marker on the X chromosome, wherein the selectable marker is mutant on the active X chromosome and wild-type on the inactive X chromosome. The female cell does not express Nanog mRNA, and preferably does not express other ES cell markers. Alternatively, the selectable marker can be one that is integrated into the inactive X chromosome, e.g., of a transgenic animal or cell, such that marker expression is only observed if the X is re-activated. Such a marker can include, for example, any positive selectable marker. A preferred embodiment of this alternative uses GFP (see the Examples herein below).

In other preferred embodiments, the selectable marker is, for example, hypoxanthine phosphoribosyltransferase (Hprt). Female cell lines heterozygous for Hprt include, for example, DR4 mouse cells (see ATCC SCRC-1045), the human TK6 lymphoblastoid cell line (ECACC 87020507), fibroblasts described by Rinat et al., 2006, Mol. Genet. Metab. 87: 249-252, and lymphocytes described by Rivero et al., 2001, Am. J. Med. Genet. 103: 48-55 and by Hakoda et al., 1995, Hum. Genet. 96: 674-680, each of which is incorporated herein by reference.

In the next step, the female cell is re-programmed to a pluripotent phenotype as described herein for other aspects of the invention.

Re-programmed cells are then cultured with a selection agent, wherein the reactivation of the inactive X chromosome permits the expression of a wild-type selectable marker and permits cell survival in the presence of the selection agent. The surviving cells are induced pluripotent stem cells.

In one embodiment of this aspect, the method further comprises the step of testing a cell surviving in the presence of the selection agent for the expression of a stem cell marker. The stem cell marker can be selected, for example, from the group consisting of SSEA1, CD9, Nanog, Fbx15, Ecat1, Esg1, Eras, Gdf3, Fgf4, Cripto, Dax1, Zpf296, Slc2a3, Rex1, Utf1, and Oct4.

In another embodiment, re-programming comprises one of the following: introducing nucleic acid sequences encoding the transcription factors Oct4, Sox2, c-Myc and Klf4 to the cell, the sequences operably linked to regulatory elements for the expression of the factors; introducing one or more protein factors that re-program the cell's differentiation state; and contacting the cell with a small molecule that induces a re-programming of the cell's differentiated state.

In another embodiment, the method further comprises the step of introducing cells that survive in the presence of the selection agent into nude mice and performing histology on a tumor arising from the cells, wherein the growth of a tumor comprising cells from all three germ layers further indicates that the cells are pluripotent stem cells.

In another embodiment, the cell is derived from a cell line.

In another embodiment, the cell is heterozygous for a mutant Hprt gene on the X chromosome.

In another embodiment, the cell carries a wild-type Hprt gene on the X chromosome that is inactive before the re-programming and a mutant, non-functional Hprt gene on the X chromosome that is active before the re-programming.

In another embodiment, the cell is resistant to 6-thioguanine before re-programming.

In another embodiment, the selection agent comprises HAT medium.

In another aspect, a method of selecting induced pluripotent stem cells is provided. The method comprises the following steps: (a) providing a female cell which carries an X-chromosome-linked reporter gene that is subject to silencing by X inactivation; wherein the female cell does not express Nanog mRNA when measured by RT-PCR; (b) the cell is re-programmed to a pluripotent phenotype; (c) the cell is then cultured after the re-programming step; and (d) a clone of a cell is isolated from the culture which expresses the X-chromosome-linked reporter. The expression of the reporter is indicative that the clone comprises induced pluripotent stem cells.

In one embodiment, the method further comprises the step of testing cells of the clone for the expression of a stem cell marker. The stem cell marker can be selected, for example, from the group consisting of SSEA1, CD9, Nanog, Fbx15, Ecat1, Esg1, Eras, Gdf3, Fgf4, Cripto, Dax1, Zpf296, Slc2a3, Rex1, Utf1, and Oct4.

In another embodiment, the method further comprises the step of introducing cells that express the reporter into nude mice and performing histology on a tumor arising from the cells. The growth of a tumor comprising cells from all three germ layers further indicates that the cells are pluripotent stem cells.

Selection of pluripotent stem cells by selecting for cells that have undergone X-reactivation can provide a system for screening for, e.g., small molecule modulators of the re-programming step, e.g., small molecules that facilitate the re-programming. Alternatively, the pluripotent stem cells derived in this manner provide for screening assays for small molecule or other modulators of the re-differentiation of the stem cells to desired phenotypes.

This invention is further illustrated by the following examples which should not be construed as limiting.

### EXAMPLES

Ectopic expression of the transcription factors Oct4, Sox2, c-Myc, and Klf4 is sufficient to confer a pluripotent state upon the fibroblast genome, generating induced pluripotent stem (iPS) cells. It remains unknown if nuclear reprogramming induced by these four factors can globally reset epigenetic differences between differentiated and pluripotent cells. Here, using novel selection approaches, iPS cells have been generated from fibroblasts to characterize their epigenetic state. Female iPS cells showed reactivation of a somatically silenced X chromosome and underwent random X inactivation upon differentiation. Genome-wide analysis of two key histone modifications indicated that iPS cells are highly similar to ES cells. Consistent with these observations, iPS cells gave rise to viable high degree chimeras with contribution to the germ line. These data show that transcription factor-induced reprogramming leads to the global reversion of the somatic epigenome into an ES-like state. These results provide a paradigm for studying the epigenetic modifications that accompany nuclear reprogramming, and suggest that abnormal epigenetic reprogramming does not pose a problem for therapeutic applications of iPS cells. These data are now published by Maherali, N., et al (2007) Cell-Stem Cell 1:55-70, which is incorporated herein in its entirety.

### Experimental Procedures

### Derivation of Fibroblasts

The Nanog-GFP-iresPuro construct (Hatano et al., 2005) was targeted into male V6.5 ES cells, correctly targeted clones were confirmed by standard Southern blot analysis, and mice were generated. Oct4-neomycin/hygromycin selectable MEFs were obtained from intercrosses between Oct4-neomycin mice with pgk-Hygromycin mice. TTFs carrying the X^{GFP} and the Oct4-neo allele were obtained from intercrosses between Oct4-Neo and X-linked GFP mice (Hadjantonakis et al., 1998). Inducible Oct4 mice have been described previously (Hochedlinger et al., 2005). MEFs were derived from embryos at embryonic day 14.5, and TFTs from up to one week old mice.

### Retrovirus Production and Infection of MEFs

cDNAs for Oct4, Sox2, c-MYC (T58A mutant), and Klf4 were cloned into the retroviral pMX vector and transfected into PlatE packaging cell line (Morita, S., Kojima, T., and Kitamura, T. (2000) Gene Ther 7, 1063-1066) using Fugene (Roche). At 48 h post-transfection, viral supernatants were used to infect target MEFs cultured in ES media. Two to three rounds of overnight infection were performed, cells were split onto a layer of irradiated feeders after 7 days and selected with 1 ug/mL puromycin (Sigma) or 300 ug/mL G418 (Roche) at indicated times.

### Cell Culture and In Vitro Differentiation

iPS cells and ES cells were grown on irradiated murine embryonic fibroblasts (feeders) and in standard ES media (DMEM supplemented with 15% FBS, non-essential amino acids, L-glutamine, penicillin-streptomycin, beta-mercaptoethanol, and with 1000 U/mL LIF). To label the 2D4 iPS cells for blastocyst injections, cells were electroporated with a Rosa-GFP-Neo targeting vector and verified by Southern blot analysis. To generate subclones from X^{GFP}/X Oct4-Neo iPS cells, cells were electroporated with a linearized pgk-Hygro plasmid. Selection was initiated 24 h post-pulse with G418 (300 ug/mL) or hygromycin (140 ug/mL), respectively. To study the state of the X chromosome, iPS cells were passaged once in ES media onto gelatin-coated dishes to reduce the number of feeder cells, and differentiation was induced with 40 ng/ml all-trans retinoic acid in ES media lacking LIF. To analyze randomness of X inactivation, differentiation was induced upon EB formation.

To isolate oocytes, the female chimera was super-ovulated with PMS and hCG and oocytes were isolated 13 hours after the hCG injection. To induce parthenogenetic activation, oocytes were incubated in Calcium-free CZB media supplemented with 10 mM strontium chloride and 5 ugml⁻¹ cytochalasin 13 for five hours followed by cultivation in KSOM media at 37 C, 5% CO₂.

### Southern Blot Analysis for Global DNA Methylation

10 μg of genomic DNA was digested with HpaII or MspI, and fragments were separated on a 0.8% agarose gel. DNA was blotted onto HybondXL membrane (Amersham Biosciences) and hybridized with the pMR150 probe as previously described (Meissner, A., Gnirke, A., Bell, G. W., Ramsahoye, B., Lander, E. S., and Jaenisch, R. (2005). Nucleic Acids Res 33, 5868-5877).

### Bisulfite Sequencing

Bisulfite treatment of DNA was performed using the EpiTect Bisulfite Kit (Qiagen) according to manufacturer instructions. Primer sequences were as previously described; Oct4 Blelloch, R., Wang, Z., Meissner, A., Pollard, S., Smith, A., and Jaenisch, R. (2006). Stem Cells 24(9):2007-13) and Nanog (Takahashi and Yamanaka, 2006). Amplified products were purified using gel filtration columns, cloned into the pCR2.1-TOPO vector (Invitrogen), and sequenced with M13 forward and reverse primers.

### RT-PCR Analysis

To test expression of pluripotency genes from the endogenous locus, total RNA was treated with the DNA-free Kit (Ambion, Austin, Tex.) and reverse transcribed with SuperScript First-Strand Synthesis System (Invitrogen) using oligo dT primers according to manufacturer instructions. All primer sequences are shown in Table 3.

### Western Analysis, Immuno- and AP Staining

Antibodies used in the methods described herein are listed in Table 3. Alkaline phosphatase staining was performed using the Vector Red substrate kit (Vector Labs). Immunostaining was done according to Plath et al (2003).

### FISH Analysis

FISH was performed as described previously (Panning, B., Dausman, J., and Jaenisch, R. (1997) Cell 90, 907-916). Xist, Tsix and Pgk1 double stranded DNA probes were generated by random priming using Cy3-dUTP (Perkin Elmer) or FTIC-dUTP (Amersham) and Bioprime kit reagents (Invitrogen) from a Xist cDNA template and a genomic clone containing 17 kb of Pgk1 sequences, respectively. Strand specific RNA probes to specifically detect either Tsix and Xist were generated by in vitro transcription in the presence of FITC UTP from Xist exon 1 and exon 6 templates. When immunofluorescence was followed by FISH, cells were fixed with 4% PFA before the FISH procedure started, and the blocking buffer contained 1 mg/ml tRNA and RNAse inhibitor.

### Cell Fusion

Four million iPS cells were combined with four million MEFs and fused with PEG-1500 (Roche) according to manufacturer's directions. Selection was initiated 24 h post-fusion using puromycin (1 ug/mL) and hygromycin (140 ug/mL). For experiments involving Neo selection, G418 was used at 300 ug/mL. Cell cycle analysis was performed on a FACS Calibur (BD) using propidium iodide; signal area was used as a measure of DNA content.

### Chromatin Immunoprecipitation (ChIP) and Microarray Hybridization

Genome wide chromatin analysis ChIP was performed with about 1 million cells following the protocol on www.upstate.com. 10 ng of each immunoprecipitated sample and corresponding inputs were amplified using the Whole Genome Amplification Kit (Sigma), and 2 ug of amplified material was labeled with Cy3 or Cy5 (Perkin Elmer) using the Bioprime Kit (Invitrogen). Hybridization onto the mouse promoter array (Agilent G4490), washing, and scanning were carried out according to the manufacturers instructions. Probe signals (log ratio) were extracted using the Feature extraction software, normalized using Lowess normalization of the Chip Analytics software, and statistically analyzed as described herein.

### Whole Genome Expression Analysis

Duplicate samples of 500 ng of RNA from V6.5 ES cells, female NGiP MEFs, puromycin-selected 2D4 iPS cells, and puromycin-selected control NGiP ES cells were amplified and labeled with Cy3 using the Agilent low RNA amplification and one color labeling kit according to manufacturer's instructions. Labeled RNA was hybridized to the Agilent Mouse whole genome array (G4122F), and analyzed.

### Flow Cytometry

For chimera analysis, spleen, thymus, and bone marrow were isolated as previously described (Ye, M., Iwasaki, H., Laiosa, C. V., Stadtfeld, M., Xie, H., Heck, S., Clausen, B., Akashi, K., and Graf, T. (2003) Immunity 19, 689-699); cells were stained with antibodies and analyzed by FACS. Oct4-Neo X^{GFP}/X tail tip fibroblasts were sorted at two consecutive passages and reanalyzed to verify a pure GFP negative population. Upon EB differentiation, cells were sorted into GFP+/GFP− populations and used for FISH analysis. Cells were acquired on a BD FACS ARIA (BD Pharmingen) and data analyzed using FlowJo software (Tree Star, Inc.).

### Teratoma Formation

Two million cells for each line were injected subcutaneously into the dorsal flank of isoflurane-anesthetized SCID mice. Teratomas were recovered three to four weeks post-injection, fixed overnight in 10% formalin, paraffin embedded and processed with hematoxylin and eosin or with specific antibodies.

### Histology and Immunohistochemical Analysis of GFP Expression in Chimeric Mice

Frozen sections were generated by subsequently incubating tissues in 4% PFA and 20% sucrose, followed by embedding in OCT compound and sectioning on a cryostat (10 μm thickness). Sections were coverslipped with Vectashield mounting media and DAPI, then visualized directly for GFP signal.

### Efficiency of iPS Cell Generation

Viral packaging PlatE cells were either transfected with 12 ug of the four factors (3 ug each factor) or with 12 ug total of a 1:3 mix of GFP vector: empty vector. Nanog-GFP MEFs were seeded at 50% confluence and infected with supernatant from the packaging cells. Seven days after infection, four factor-infected cells were split 1:2 onto irradiated feeders and placed either under selective (1 ug/mL puromycin) or non-selective conditions. GFP-infected cells were counted (5.3×10⁶) and analyzed by FACS. The percentage of GFP+ cells (15%) was taken to be the frequency of infection with one factor, thus the frequency for all four factors as 0.15⁴, giving a theoretical yield of ˜2700 colonies. After four weeks under selective conditions, 20 AP positive puro-resistant colonies emerged, giving an efficiency of ˜0.74%. Under non-selective conditions, ˜240 colonies emerged, giving an efficiency of ˜9%.

### Chromatin Immunoprecipitation

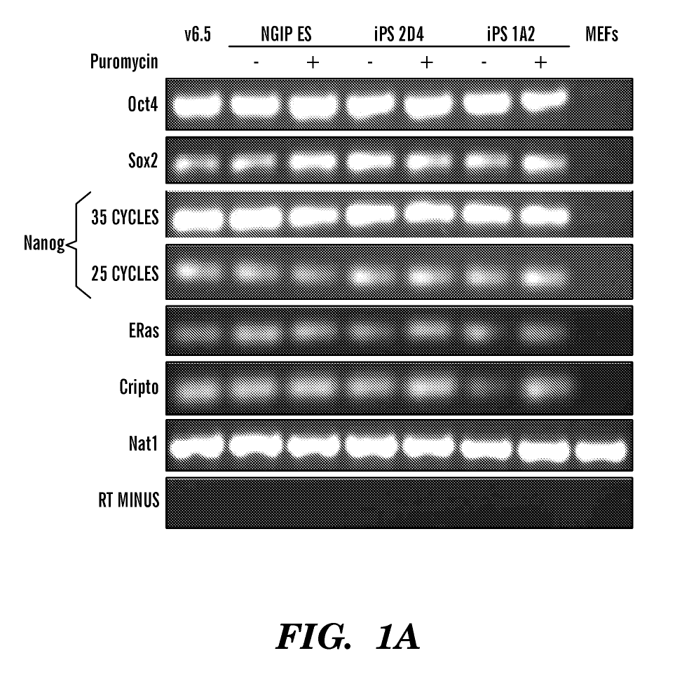

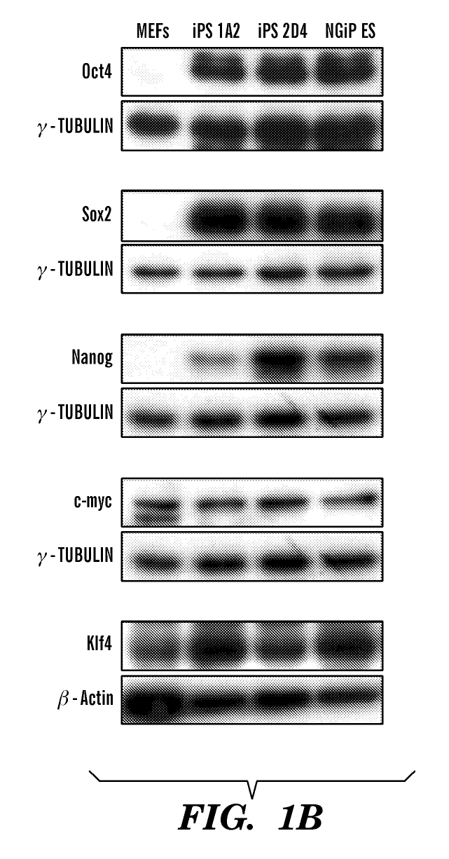

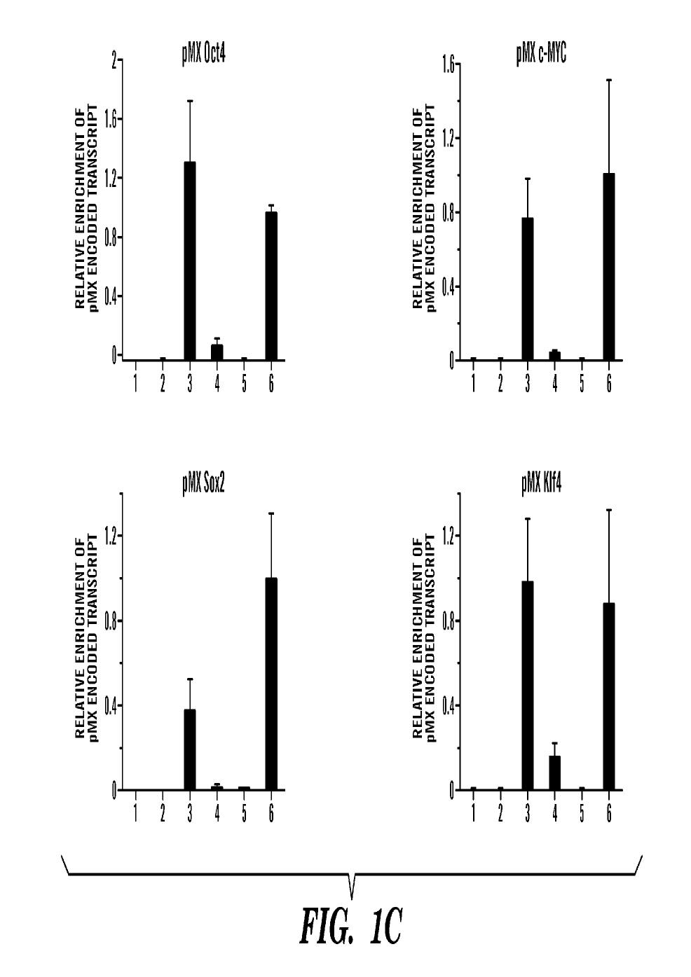

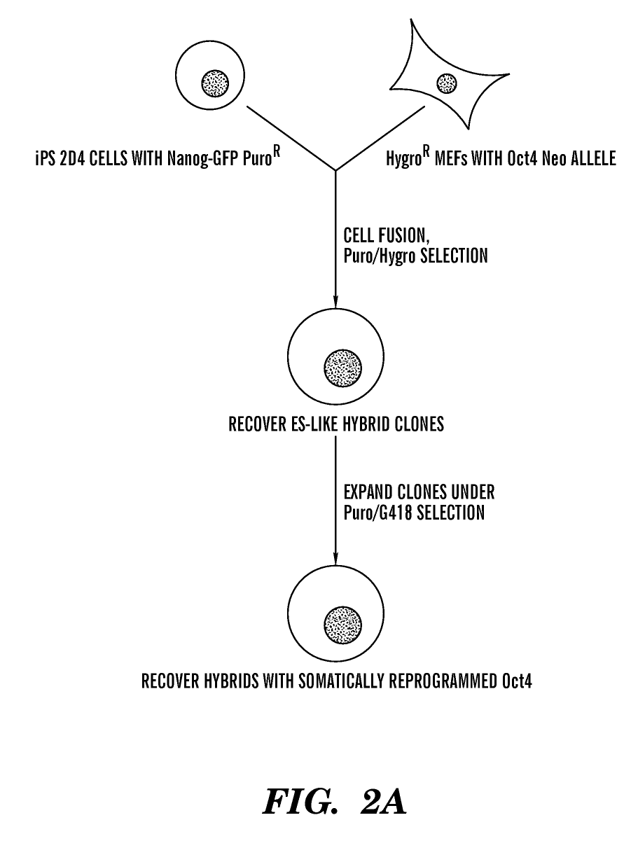

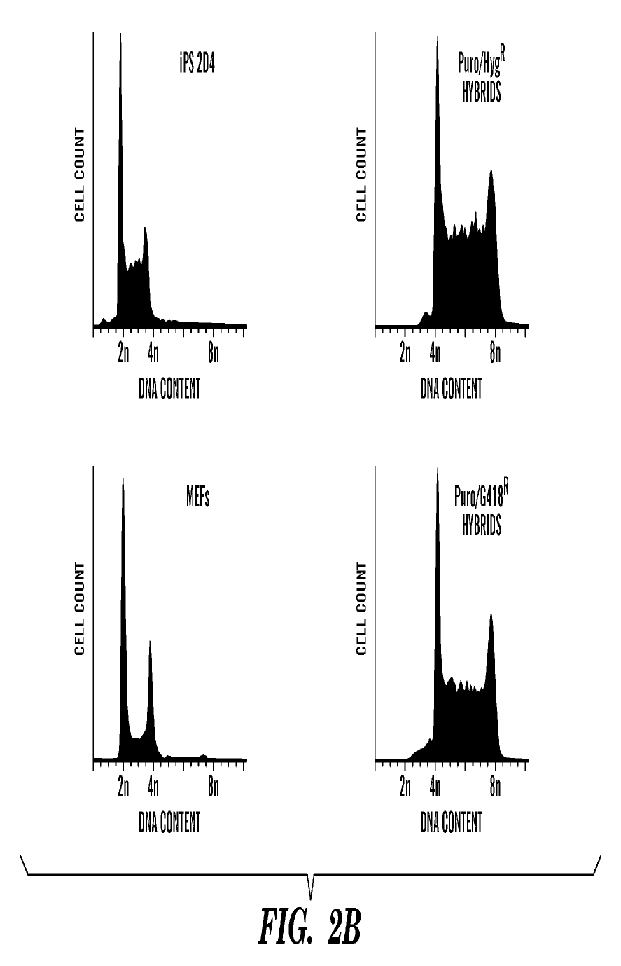

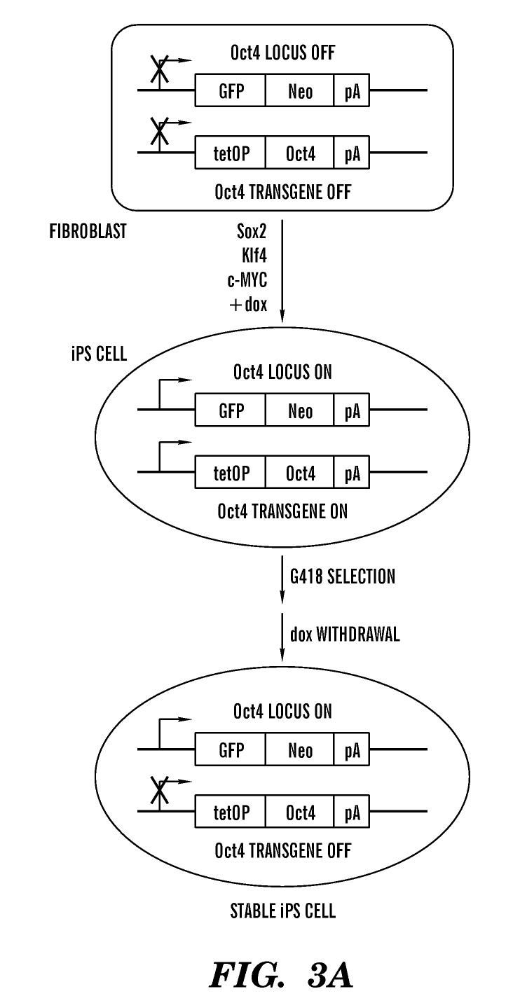

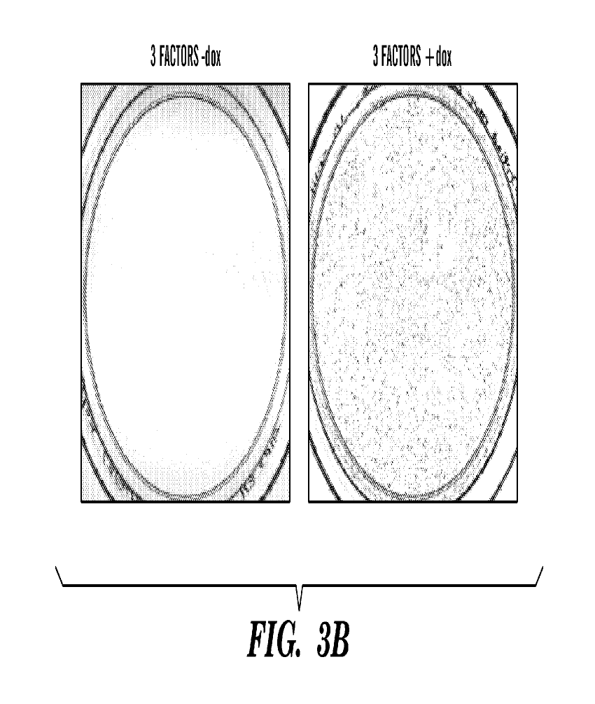

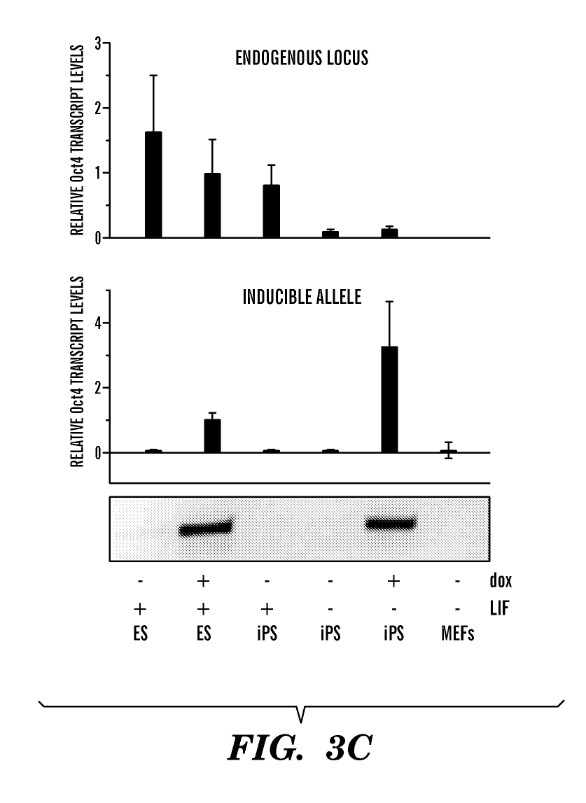

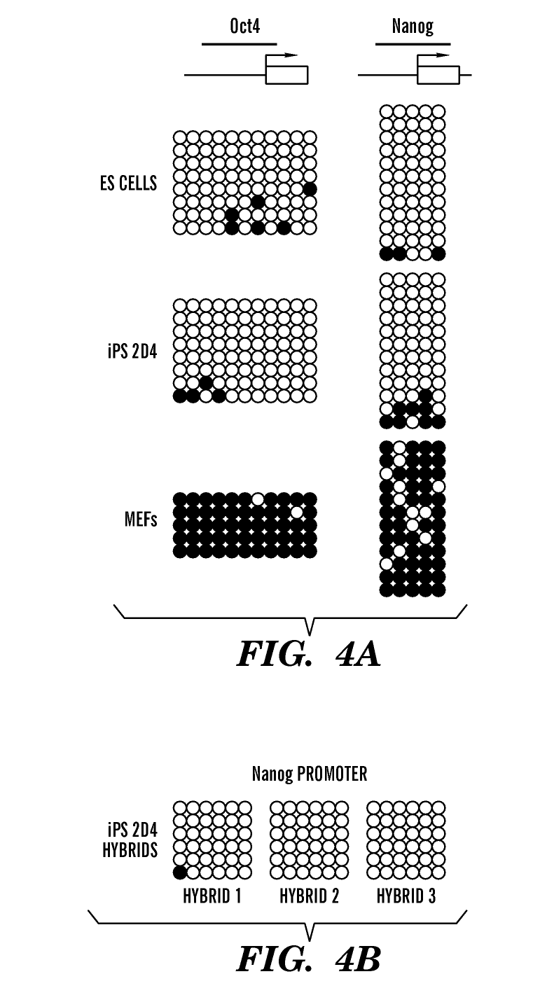

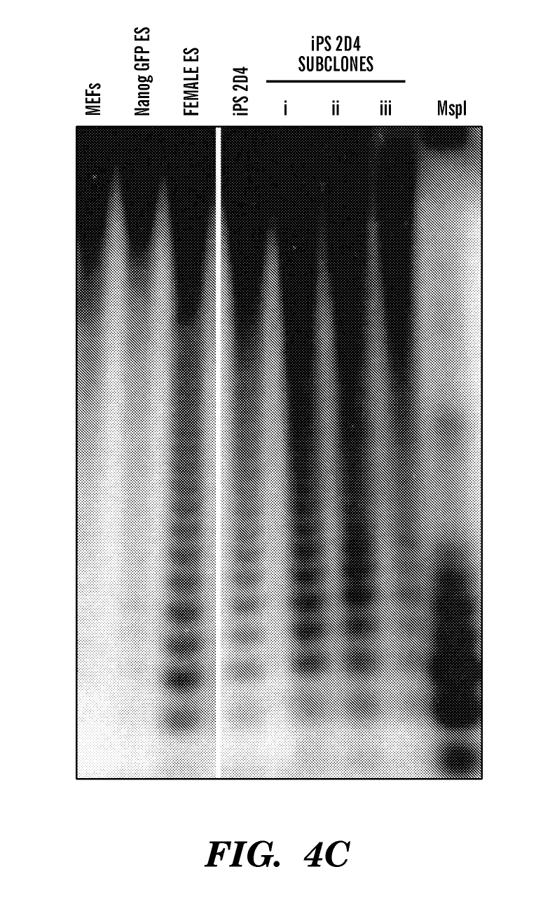

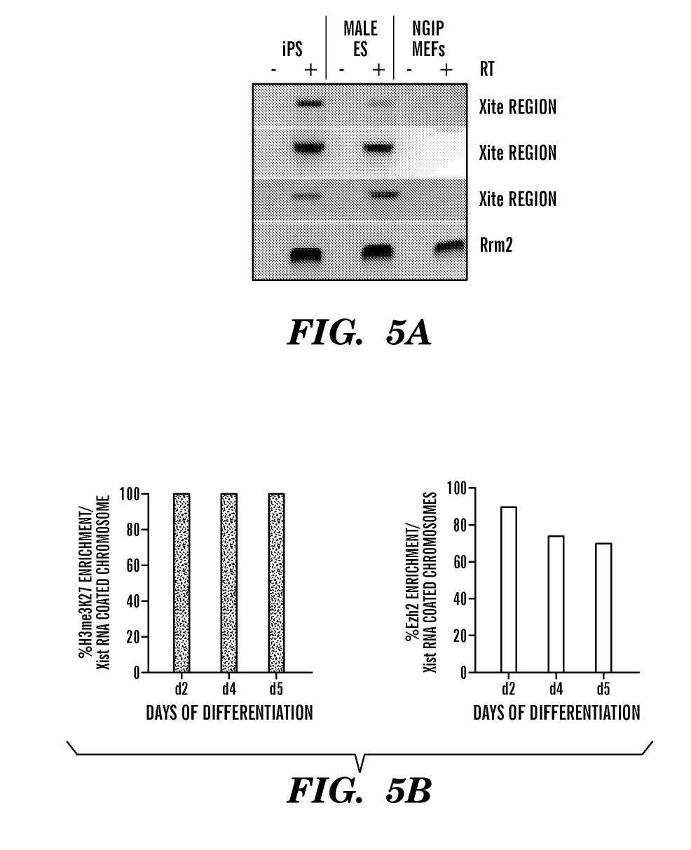

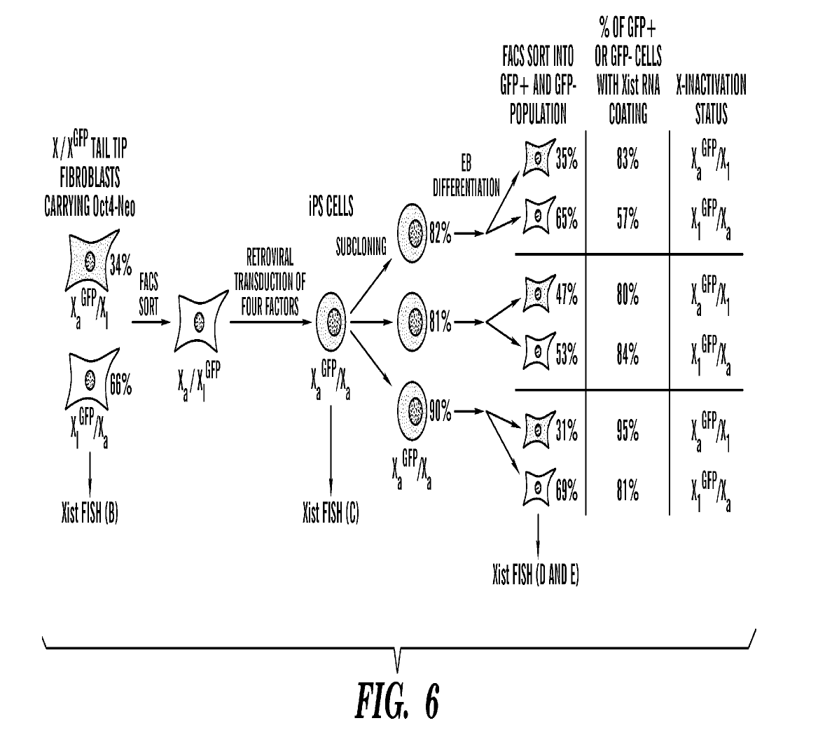

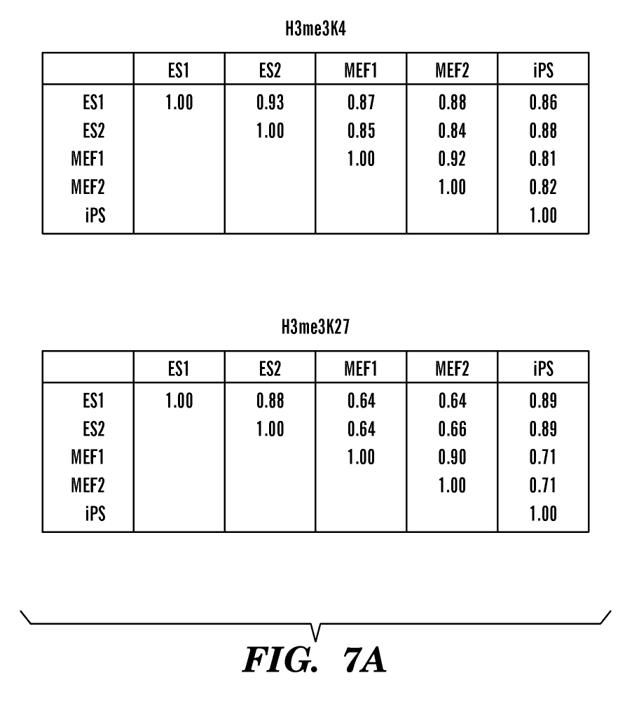

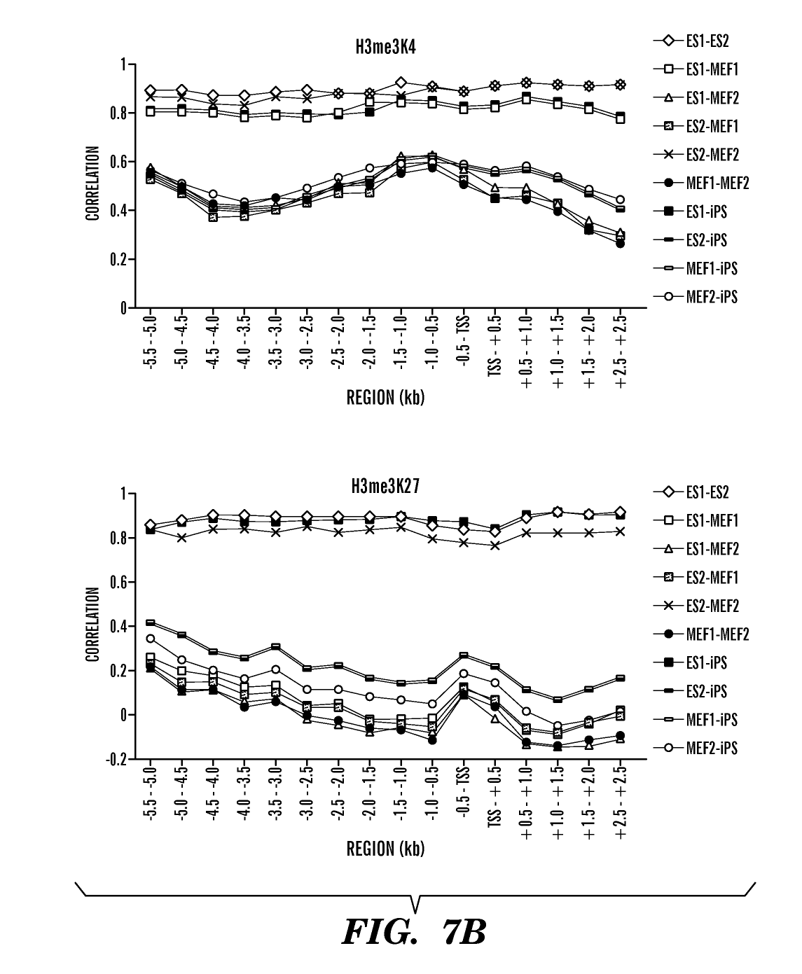

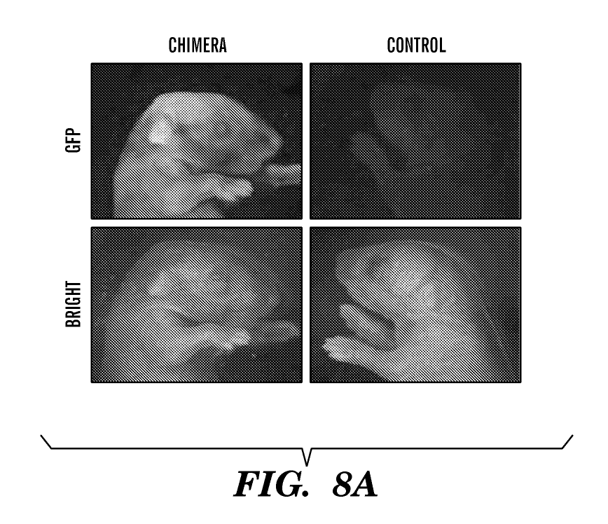

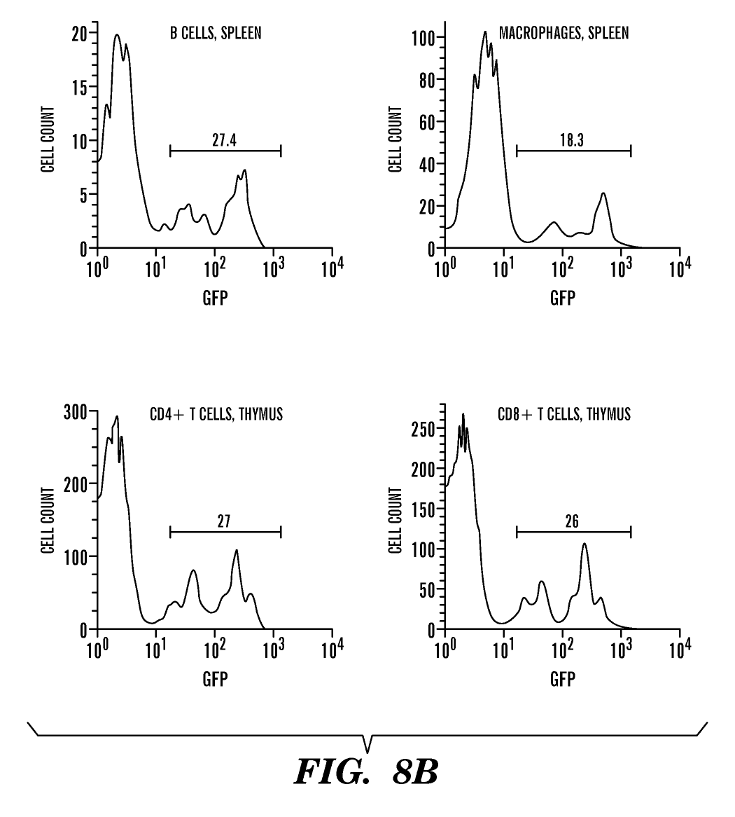

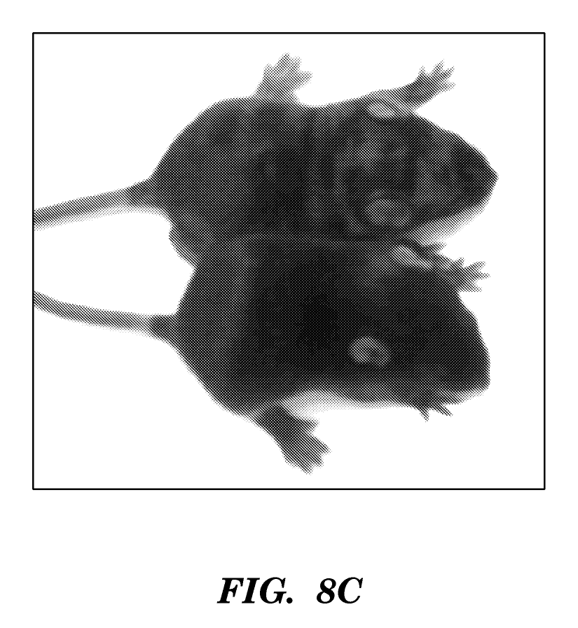

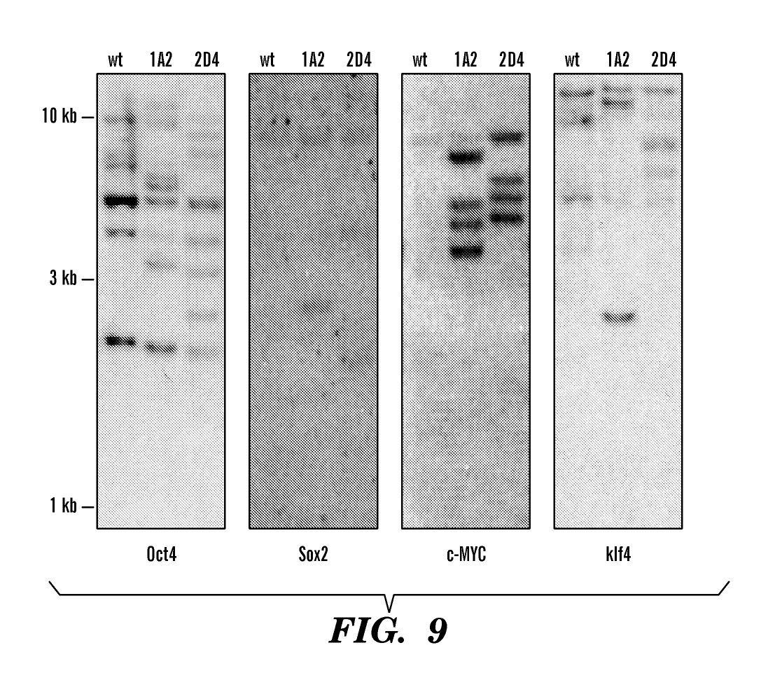

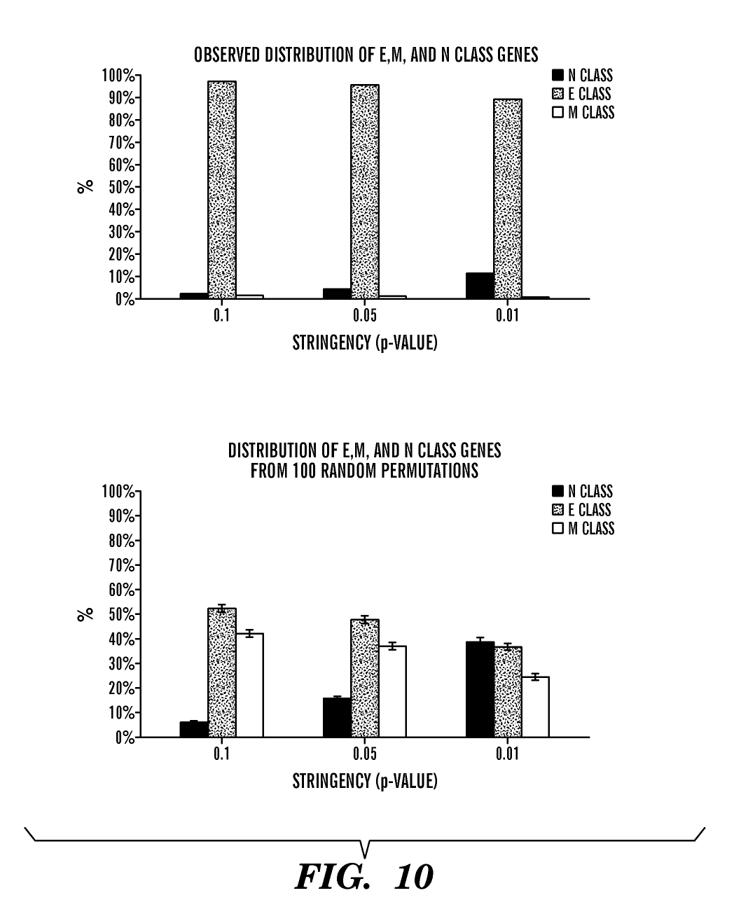

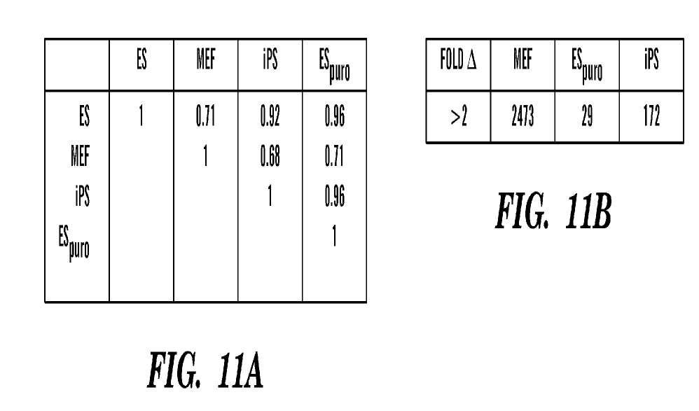

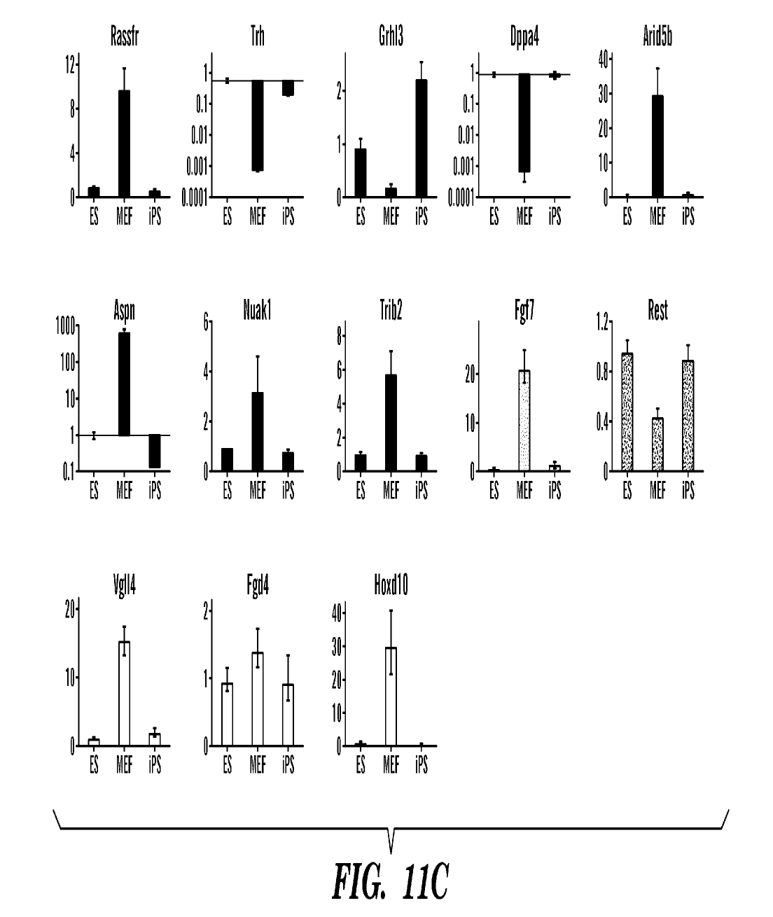

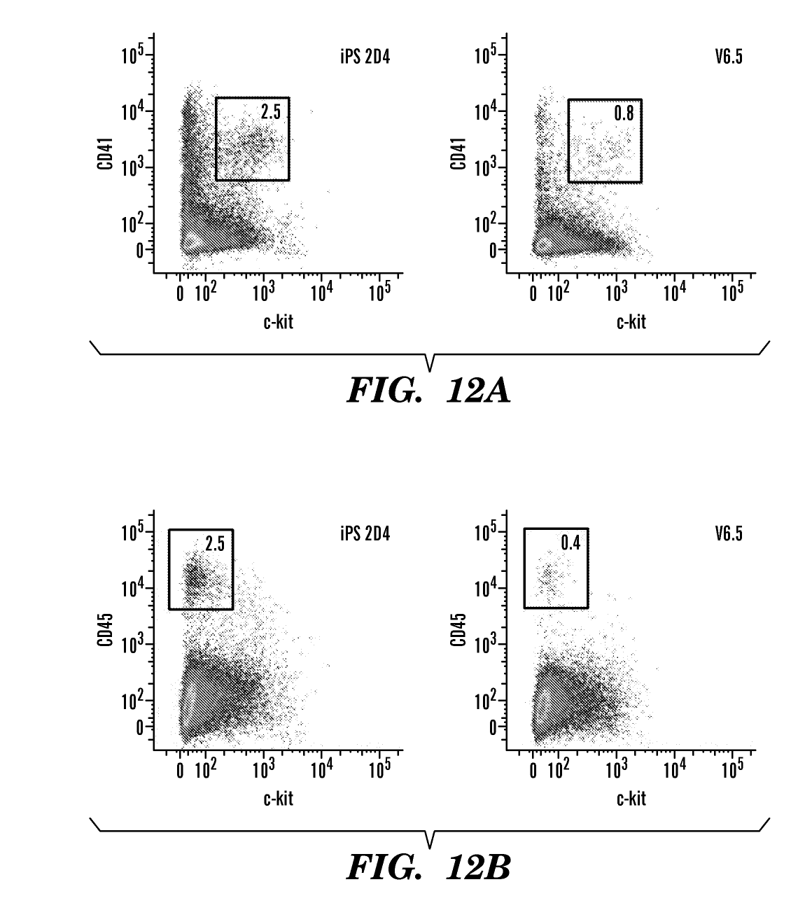

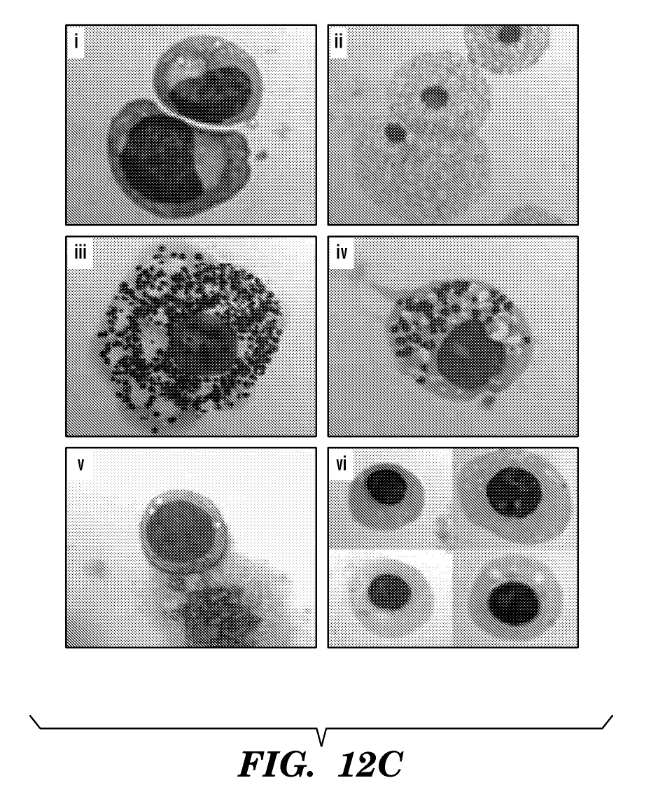

The feeder dependent male ES cell line V6.5 (129/B16), the feeder-independent male ES cell line E14 (129/ola), and primary male and female MEFs derived from 129/B16 mice were used, as well as the 2D4 iPS line grown in the presence of puromycin. In ease of the V6.5 and 2D4 cells, to reduce fibroblast contamination, the last passage of the cells was done without adding additional feeder cells. The cells maintain their undifferentiated state under these conditions (FIG. 1 and data not shown). Cells were crosslinked with formaldehyde for 10 min at room temperature, subsequently lysed in 10 mM Tris-EDTA pH 8.0 with 1% SDS, and sonicated on ice 6 times at 15 second pulses interrupted by 45 second pauses. Clarified sheared chromatin was immunoprecipitated with antibodies to H3me3K4 (Abeam 8580) or H3me3K27 (Upstate 07-449) overnight at 4 C, collected with protein A beads for 2 hours, washed twice for 5 min and eluted with buffers (recipes on the Upstate website). Eluates were reverse crosslinked, RNAse and proteinase K treated, and DNA was purified using the Qiagen PCR purification kit. ChIP with rabbit IgG antibody did not find any enrichment (data not shown).

### Statistical Methods for the Analysis of Genome-with Histone Methylation Data

Average probe signals were extracted in a 500 bp window-step-wise manner. 16339 genes were selected based on the criteria that at least 50% of the regions are covered by probes in a 500 bp-window manner. Genes with significant difference of H3me3K4 and H3me3K27 patterns between ES cells and MEF cells were filtered as signature genes. For each gene, the difference of histone modification patterns between two cell types was defined by the Euclidean distance of the 16-window signal vectors. Self-distance of the two ES cell lines (dist E14 vs. V6.5) and the two primary MEF cell lines (dist male (M) vs. female (F)) was pooled to generate the null distribution, assuming that the differences between two ES cell lines or two MEF cell lines are small. Genes encoded on the X and Y chromosomes were excluded from the analysis. For all signature genes, the distance of any ES-MEF pair (dist E14 vs. M; dist E14 vs. F, dist V6.5 vs. M; dist V6.5 vs. F) has to be greater than the pre-defined signature-gene threshold (SigT) which is the 99% quantile of the null distribution (corresponding to p-value of 0.01).

To classify the methylation pattern of signature genes in the 2D4 line into Es-like genes (E class), MEF-like genes (M class), and Neutral genes (N class; genes that do not show significantly stronger preferences to either ES cells or MEFs), the average distances between 2D4 and the ES cells (dist 2D4 vs. ES) and the average distances between 2D4 and MEFs (dist 2D4 vs. MEF) were computed. A Preference Score PS_{—}2D4=(dist 2D4 vs. ES-dist 2D4 vs. MEF), was used as an index of how strongly the histone methylation pattern of a particular gene in 2D4 cells “prefers” and presumably mimics the pattern of ES cells. Again, a null distribution of the PS was generated in the following way. The data set of each ES cell line was compared with that of the other ES line and that of MEFs. The PS_ES (dist E14 vs. V6.5-dist E14 vs. MEF) and (dist V6.5 vs. E14-dist V6.5 vs MEF) from all 16339 genes were computed and pooled. A 95% quantile was used as E class threshold (ET). Any signature genes with PS_{—}2D4 greater than the ET were called “M class”. M threshold (MT) and the E class were defined similarly. Genes for which PS-2D4 falls between MT and ET were called “N class”. The Pearson correlation coefficient of the methylation data for each 500 by window within the 8 kb region between different cell types was calculated using the correl function in MS Excel.

### Gene Expression Analysis

Expression data were extracted using the Feature Extraction software (Agilent). Raw data was log 2 transformed and signals from multiple probes for the same gene were averaged. Each array was normalized so that the mean was 0 and standard deviation was 1. Data from replicate experiments were averaged. Genes with a two fold change in expression between MEFs and ES cells were selected, resulting in the identification of 2473 genes that are most dissimilarly expressed between these two cell types (out of 33376 total genes). Unbiased hierarchical clustering was employed to group the expression pattern for these 2473 genes across ES cells, MEFs, puro selected NGiP ES cells and iPS cells. In addition, the expression pattern for the signature genes was computed as a ratio of ES and MEF or iPS and MEF and plotted along with the methylation data.

### Example 1

### Generation of iPS Cells Using Nanog-Selectable Fibroblasts

Female mouse embryonic fibroblasts (MEFs) carrying a GFP-IRES-Puro cassette in the endogenous Nanog locus, referred to as Nanog-GFP-puro (Hatano, S. Y., Tada, M., Kimura, H., Yamaguchi, S., Kono, T., Nakano, T., Suemori, H., Nakatsuji, N., and Tada, T. (2005) Mech Dev 122, 67-79), were retrovirally infected with cDNAs encoding Oct4, Sox2, c-MYC—T58A mutant, which stabilizes the protein (Sears, R., Nuckolls, F., Haura, E., Taya, Y., Tamai, K., and Nevins, J. R. (2000) Genes Dev 14, 2501-2514)—and Klf4. In contrast to the previously reported Fbx 15 selection, which was applied three days after infection (Takahashi and Yamanaka, 2006), selection for Nanog expression at three days post-infection resulted in no colonies, suggesting different reactivation kinetics of the Fbx15 and Nanog genes. When selection was applied seven or more days following infection, resistant colonies reproducibly emerged. Of the five lines that were expanded (see Table 1), two lines maintained homogeneous cultures that appeared identical to ES cells and expressed the ES cell surface markers SSEA1 and CD9 (data not shown). In contrast, the other three clones gave rise to heterogeneous cultures after multiple passages, which contained both an ES-like population and a separate population of small round, rapidly dividing cells. FACS sorting for Nanog-GFP, SSEA-1, and CD9, followed by sub-cloning, was sufficient to eliminate these round cells, suggesting that this population was distinct from the ES-like cells. Interestingly, the onset of selection for the two homogeneous cell lines occurred at three weeks post-infection, while the heterogeneous lines had undergone selection at one week post-infection, suggesting that delayed selection may be advantageous for obtaining a more pure population of iPS cells.

Subsequent studies focused on the homogeneous ES-like cell line 2D4 and the re-sorted and subcloned line 1A2, which are referred to herein as iPS cells. Southern blot analysis of retroviral integration sites revealed the presence of all four retrovirally-encoded genes in both iPS lines, and a test for genomic imprinting confirmed that the iPS cells were not derived from rare primordial germ cells that may have been present in the fibroblast culture (FIG. 9). In contrast to Fbx15-selected iPS cells (Takahashi and Yamanaka, 2006), Nanog-selectable iPS cells exhibited feeder-independent growth, as they maintained an ES-like morphology, Nanog expression, and alkaline phosphatase (AP) activity in the absence of feeders and puromycin selection (data not shown). Withdrawal of LIF resulted in the expected differentiation into GATA-4-expressing cells resembling primitive endoderm (data not shown), and differentiation was accompanied by a loss of Nanog expression (data not shown). RT-PCR analysis indicated expression of Oct4 and Sox2 from the endogenous loci, along with the other ES cell markers Nanog, ERas, and Cripto (FIG. 1A). Quantitative PCR analysis for the four retrovirally expressed genes showed strong expression in fibroblasts infected with the individual retroviruses but efficient silencing in homogenous iPS cells (FIG. 1C). Protein levels for Oct4, Sox2, c-Myc and Klf4 were similar between iPS cells and control ES cells (FIG. 1B), and immunofluorescence showed that Oct4 and Sox2 were efficiently downregulated upon retinoic acid-induced differentiation, demonstrating that the virally encoded transcription factor genes remained effectively silenced in differentiated cells (data not shown). Injection of 2D4 iPS cells into SCID mice gave rise to teratomas containing cell types representative of the three germlayers, confirming their pluripotency (data not shown). These data indicate that retrovirally expressed Oct4, Sox2, c-MYC and Klf4, in combination with selection for Nanog reactivation, can yield iPS cells that share many properties with ES cells.

### Example 2

### Nanog-Selectable iPS Cells Confer an Es Cell-Like Phenotype Upon Somatic Cells

To determine whether Nanog-selectable iPS cells possess functional attributes similar to ES cells, the ability to impose an ES-like phenotype upon somatic cells in the context of cell fusion was tested. Cells from the puromycin resistant 2D4 iPS cell line with hygromycin-resistant MEFs (FIG. 2A). Two weeks after fusion, seven double-resistant tetraploid hybrid clones that had an ES cell-like morphology and continued to express Nanog-GFP (FIG. 2B and data not shown) were recovered. One hybrid colony was recovered when control Nanog-GFP-puro ES cells were fused with hygromycin-resistant MEFs. To test pluripotency, hybrid cells were injected into immunocompromised mice; after four weeks, teratomas containing cell types representative of all three germ layers were isolated (data not shown).

As a test for reprogramming of the somatic cell genome, the fusion experiment was repeated with MEFs that contained both a constitutive hygromycin resistance gene and a neomycin selectable marker under the control of the endogenous Oct4 locus (referred to as Oct4-Neo allele). No clones could be obtained if G418 was used in the initial selection process, suggesting that the reprogramming of the somatic cell Oct4 locus, like that of the endogenous Nanog locus, is a gradual process. Therefore, the puromycin/hygromycin resistant hybrids were expanded before subjecting them to puromycin/G418 selection to test for reactivation of the somatic Oct4 gene. All puromycin/hygromycin resistant colonies were viable under puromycin/G418 selection, indicating that the somatic genome had been reprogrammed at the endogenous Oct4 locus (data not shown). These results show that Nanog-selected cells, similar to ES cells, carry reprogramming activity and can confer an ES-like state upon a somatic cell genome.

### Example 3

### Ectopic Oct4 Expression is Dispensable for the Maintenance of iPS Cells

Fbx15-selected 2D4 iPS cells showed persistent retroviral expression of Oct4 and Sox2 with negligible expression from the respective endogenous loci, suggesting a continuous requirement for the exogenously provided factors to maintain the self-renewal and pluripotency of iPS cells (Takahashi and Yamanaka, 2006). To corroborate the gene expression data that suggested efficient retroviral gene silencing in iPS cells, it was decided to genetically test whether continuous Oct4 expression is required for the maintenance of iPS cells by using fibroblasts carrying a doxycycline-inducible Oct4 transgene in their genome (Hochedlinger, K., Yamada, Y., Beard, C., and Jaenisch, R. (2005) Cell 121, 465-477) (FIG. 3A).

To initially determine whether colonies could be obtained using the Oct4 inducible system, Oct4-inducible MEFs were infected with Sox2, c-MYC, and Klf4 retroviruses without any selection. In the absence of doxycycline, no AP positive colonies were recovered, while in the presence of doxycycline several hundred AP positive colonies emerged, indicating a strict dependence on transgenic Oct4 expression for the establishment of AP positive colonies (FIG. 3B). Subsequently, iPS cells were generated from tail tip fibroblasts (TTFs) carrying both the Oct4 inducible allele and the Oct4-Neo allele to verify the reprogrammed state of resultant cells (FIG. 3A). Target cells were infected with Sox2, c-MYC, and Klf4 in the presence of doxycycline. Based on the previous observation that a late onset of drug selection was advantageous, it was attempted to establish iPS colonies based solely on ES cell-like morphology without initial selection. 48 individual ES-like colonies were picked at three weeks post-infection, two of which grew into stable ES cell-like lines in the continued presence of doxycycline. Following replating into G418 media, both cell lines survived, indicating that the endogenous Oct4 gene had been reactivated and iPS cells had been generated. Importantly, when doxycycline was withdrawn from the media, these cells could be passaged many times in the presence of G418 without changes in their growth behavior or morphology (data not shown). To exclude the possibility of viral insertion and aberrant Oct4 transgene activation in the absence of doxycycline, quantitative PCR analysis of endogenous and induced Oct4 expression was performed to analyze expression levels during differentiation and induction (FIG. 3C). Undifferentiated iPS cells showed high levels of endogenous Oct4 expression and complete absence of transgene expression. Oct4 levels declined in the absence of LW and reappeared upon administration of doxycycline, indicating differentiation-dependent downregulation of endogenous Oct4 expression and sustained responsiveness of cells to doxycycline, respectively (FIG. 3C). The ability to form well-differentiated teratomas demonstrated the pluripotency of these cells (data not shown). Thus, the endogenous Oct4 locus was sufficiently reprogrammed by the four transcription factors to maintain iPS cells in a pluripotent state in the absence of exogenous Oct4 expression.

### Example 4

### Gene Specific and Global DNA Methylation is Similar Between iPS Cells and ES Cells

Based on the ES cell-like properties of reprogrammed fibroblasts, it was asked if iPS cells had acquired an epigenetic state similar to ES cells. Reprogramming of a somatic genome by nuclear transfer or cell fusion is accompanied by epigenetic changes such as DNA demethylation of pluripotency genes at their promoter regions (Cowan et al., 2005; Tada et al., 2001). Bisulfite sequencing was used to assess the methylation status of the Oct4 and Nanog promoters, which had previously been shown to be incompletely de-methylated in Fbx15-selected iPS cells (Takahashi and Yamanaka, 2006). Both promoter elements, which were methylated in MEFs, showed de-methylation in Nanog-selected iPS cells and ES cells, suggesting proper epigenetic reprogramming of these two pluripotency genes (FIG. 4A). Furthermore, de-methylation of the Nanog promoter occurred in cell hybrids generated through fusion of iPS cells and MEFs (FIG. 4B; refer to FIG. 2), confirming that iPS cells harbor reprogramming activity and can induce epigenetic changes in differentiated cells.

Female ES cells, in contrast to male ES cells and differentiated cells, show global DNA hypo-methylation of the genome which is attributable to the presence of two active X chromosomes (Xa) (Zvetkova, I., Apedaile, A., Ramsahoye, B., Mermoud, J. E., Crompton, L. A., John, R., Feil, R., and Brockdorff, N. (2005) Nat Genet. 37, 1274-1279). Using a methylation sensitive restriction enzyme assay, global hypo-methylation of minor satellite repeats was detected in the 2D4 iPS cell line, similar to female control ES cells (FIG. 4C). These results suggest that iPS cells have obtained an epigenetic state similar to that of female ES cells.

### Example 5

### X-Inactivation in Female Nanog-Selectable iPS Cells

Global DNA hypo-methylation in iPS cells indicates that the inactive X chromosome (Xi) is reactivated in female iPS cells. X-inactivation is one of the most dramatic examples of heterochromatin formation in mammalian cells, and is regulated by two non-coding RNAs, Xist, and its antisense transcript Tsix, which are reciprocally expressed (Thorvaldsen, J. L., Verona, R. I., and Bartolomei, M. S. (2006) Dev Biol 298, 344-353). Undifferentiated female ES cells carry two Xa and express Tsix from both X chromosomes to repress Xist expression. Upon differentiation, Xist becomes strongly upregulated on the future Xi to induce silencing, while Tsix disappears and is absent in somatic cells. The Xite locus, a third locus important for X-inactivation located downstream of Tsix, is expressed in a Tsix-like pattern (Ogawa, Y., and Lee, J. T. (2003) Mol Cell 11, 731-743).

The X-inactivation status in female Nanog-GFP-puro MEFs was first assessed using fluorescence in situ hybridization (FISH) to analyze Xist RNA localization and X-linked gene expression. In agreement with the presence of an Xi, 96% of the fibroblasts carried an Xist RNA-coated X chromosome and showed expression of the Pgk1 gene from the other X chromosome (data not shown). The 2D4 iPS cell line showed a pattern of Kist, Tsix, and Pgk1 expression highly reminiscent of undifferentiated ES cells (data not shown). That is, Tsix and Pgk1 were expressed bi-allelically at high levels, and Xist RNA could not be detected, demonstrating the presence of two Xa. In addition, RT-PCR analysis detected transcripts from the Xite locus in both ES cells and 2D4 iPS cells, but not in the parental fibroblast population (FIG. 5A).

Upon initiation of X-inactivation, characteristic chromatin modifications are imposed on the future Xi that ensure stable silencing of the chromosome (Heard, E. (2005) Curr Opin Genet Dev 15, 482-489; Ng, K., Pullirsch, D., Leeb, M., and Wutz, A. (2007) EMBO Rep 8, 34-39). Immunofluorescence was used to analyze the presence of Xi-linked chromatin-modifications in iPS cells. Female Nanog-GFP-puro MEFs showed the expected frequencies of the Xi-like enrichment for histone H3 trimethylated at lysine 27, histone H4 lysine 20 mono-methylation, and for the Polycomb group (PcG) protein Ezh2, which is responsible for mediating H3K27 tri-methylation. In contrast, iPS cells, like ES cells, showed abundant and uniform nuclear staining for these chromatin marks with no Xi-like enrichment (data not shown). Together, these data indicate that four transcription factors, in combination with Nanog selection, are sufficient to induce transcriptional reactivation of the Xi, to reset the expression patterns of the three non-coding transcripts essential for regulation of X-inactivation, and to erase the chromatin modifications that are specific to the Xi.

Next, it was tested if 2D4 cells could undergo X-inactivation upon differentiation. Consistent with the ability of iPS cells to silence one of their X's, Kist RNA-coated chromosome was detected in 2D4 iPS cells undergoing retinoic acid-induced differentiation (data not shown). The Xist coated chromosome showed no overlap with RNA Polymerase II in agreement with a silent state of that X (data not shown). Furthermore, similar to differentiating female ES cells, the Xist RNA-coated X chromosome in iPS cells was almost always coincident with a region of enrichment of H3me3K27 and its methyltransferase, Ezh2, upon initiation of X-inactivation (FIG. 5B). The coincidence of Ezh2 accumulation and H3me3K27 enrichment on the Xi are hallmarks only of early phases of X-inactivation Plath, K., Fang, J., Mlynarczyk-Evans, S. K., Cao, R., Worringer, K. A., Wang, H., de la Cruz, C. C., Otte, A. P., Panning, B., and Zhang, Y. (2003) Science 300, 131-135; Silva, J., Mak, W., Zvetkova, I., Appanah, R., Nesterova, T. B., Webster, Z., Peters, A. H., Jenuwein, T., Otte, A. P., and Brockdorff, N. (2003) Dev Cell 4, 481-495). Thus, X chromosome inactivation in female iPS cells displays the same dynamics as in female ES cells.

### Example 6

### Random X Inactivation in Differentiating iPS Cells

X chromosome inactivation occurs non-randomly in extra-embryonic lineages and in early pre-implantation embryos, while it is random in the epiblast and differentiating ES cells. Analysis of X inactivation in cloned mouse embryos has shown that the somatic Xi is reprogrammed during nuclear transfer to enable random X inactivation in embryonic cells while the memory of the Xi is maintained in extra-embryonic tissues where it replaces the gametic imprint (Eggan et al, 2000). It was therefore tested whether transcription factor-induced reprogramming can erase the memory of the somatically inactivated Xi, thus enabling random X inactivation in differentiating iPS cells. Since it was not possible to distinguish between the two X chromosomes in Nanog-selectable 2D4 iPS cells, iPS cells were generated from female fibroblasts carrying an X-linked reporter transgene (X^{GFP}) with a cytomegalovirus promoter driving expression of the green fluorescent protein (GFP) (Hadjantonakis, A. K., Gertsenstein, M., Ikawa, M., Okabe, M., and Nagy, A. (1998) Nat Genet. 19, 220-222) (FIG. 6A). This reporter is subject to silencing by X-inactivation and thus permits determination of a silenced X chromosome in differentiating iPS cells. TTFs were isolated from a female mouse heterozygous for the GFP transgene and carrying the Oct4-Neo allele. Consistent with random X-inactivation in the fibroblast population, 34% of the TTF cells were GFP positive (Xa^{GFP}/Xi) and 66% of the cells were GFP negative (Xi^{GFP}/Xa) (FIG. 6A, and data not shown). Some skewing of X-inactivation was expected and likely reflected differences in the genetic backgrounds of the two X chromosomes. GFP negative cells isolated by two rounds of FACS sorting were infected with the retroviruses encoding the four transcription factors, and resulting ES-like colonies were screened for reactivation of the Xi^{GFP} based on GFP re-expression. Four entirely green colonies were isolated that, upon replating, were also found to be resistant to G418, thus indicating activation of the Oct4 locus in addition to reactivation of the silent X chromosome. An ES cell-like pattern of Xist and Tsix expression confirmed X reprogramming (data not shown).

Given that these female iPS cells, like ES cells, had a tendency to lose an X when maintained continuously in culture, Xa^{GFP}Xa iPS cells were sub-cloned to ensure that pure clonal populations of iPS cells were analyzed for randomness of X-inactivation. Differentiation of sub-clones was induced by embryoid body formation, and differentiated cells were sorted by FACS into GFP positive and GFP negative populations and analyzed by FISH (FIG. 6A). Consistent with a random pattern of X inactivation, on average 38% of the cells were GFP positive and 62% of the cells were GFP negative, and the majority of both populations had an Xist signal consistent with Xist RNA coating of the Xi (data not shown). Random X-inactivation confirms that the epigenetic marks that distinguish the Xa and Xi in somatic cells can be removed upon in vitro reprogramming and reestablished on either X upon subsequent in vitro differentiation.

### Example 7

### Global Reprogramming of Histone Methylation Patterns in iPS Cells

It was next asked if in addition to DNA de-methylation of the Oct4 and Nanog promoters and the reactivation of the Xi, the entire fibroblast genome had been epigenetically reprogrammed to an ES-like state during iPS cell derivation. Histone methylation plays a crucial role in epigenetic regulation of gene expression during mammalian development and cellular differentiation. In general, transcribed genes are associated with H3K4 tri-methylation Bernstein, B. E., Kamal, M., Lindblad-Toh, K., Bekiranov, S., Bailey, D. K., Huebert, D. J., McMahon, S., Karlsson, E. K., Kulbokas, E. J., 3rd, Gingeras, T. R., et al. (2005) Cell 120, 169-181; Kim, T. H., Barrera, L. O., Zheng, M., Qu, C., Singer, M. A., Richmond, T. A., Wu, Y., Green, R. D., and Ren, B. (2005) Nature 436, 876-880), while many silenced genes are associated with H31(27 tri-methylation (Boyer, L. A., Plath, K., Zeitlinger, J., Brambrink, T., Medeiros, L. A., Lee, T. I., Levine, S. S., Wernig, M., Tajonar, A., Ray, M. K., et al. (2006). Nature 441(7091):349-53; Lee, T. I., Jenner, R. G., Boyer, L. A., Guenther, M. G., Levine, S. S., Kumar, R. M., Chevalier, B., Johnstone, S. E., Cole, M. F., Isono, K., et al. (2006) Cell 125(2):301-13). Genome-wide location analysis for K4 and K27 tri-methylation in the Nanog-selected 2D4 iPS line, male and female MEFs, and two male ES cell lines was performed using chromatin immunoprecipitation followed by hybridization to a mouse promoter array. Probes on this array cover a region from −5.5 kb upstream to +2.5 kb downstream of the transcriptional start sites for about 16,500 genes. To determine if the 2D4 iPS line was more similar to ES cells or to MEFs, a set of genes was defined that was significantly different in the histone methylation pattern between ES cells and MEFs. At high stringency (p=0.01), 957 genes were identified as being different between ES cells and MEFs and classified as “signature” genes (see Experimental Procedures). Remarkably, in 2D4 iPS cells, 94.4% of the signature genes carried a methylation pattern virtually identical to ES cells (E class genes), while only 0.7% of the genes were methylated in a more MEF-like pattern (M class genes). The remaining 4.9% of the loci were classified as N class genes (neutral) as the differences were too small to be significant (data not shown). The majority (91%) of the iPS loci remained in the E class even when the stringency was lowered to p=0.05 to include a larger set of signature genes (data not shown). The distribution into E, M, and N genes is highly significant as confirmed by a random permutation test (FIG. 10). Genes that belonged to the non-signature class showed little or no difference in methylation pattern between MEFs, ES cells and iPS cells (data not shown), indicating that the iPS line had not acquired a completely novel epigenetic identity found neither in ES cells or MEFs. Collectively, these results indicate that in vitro reprogramming can reverse the epigenetic memory of a fibroblast genome into one highly similar to that of ES cells.

In an effort to determine if K4 and K27 methylation patterns were reset to different extents during reprogramming, Pearson correlation was calculated separately for each methylation mark for all 16,500 genes on the array (FIG. 7A). This analysis revealed that iPS cells and ES cells were as similar in their K27 methylation pattern as the two ES lines to each other, while MEFs clearly differed to the same extent from both iPS and ES cells. Interestingly, K4 methylation was more similar between all cell types, suggesting that reprogramming is mainly associated with changes in K27 rather than K4 tri-methylation. One prediction from this global analysis is that the change in K27 methylation should be prominent in the E class of signature genes. To test this, a pair-wise correlation analysis was performed between all possible cell types at 500 bp intervals along the 8 kb promoter region, resulting in 16 correlation values for each comparison (FIG. 7B). Genes classified as E genes were indeed very similar in their K4 and K27 methylation patterns between ES cells and 2D4 iPS cells along the entire analyzed region, while MEFs differed dramatically from both cell types throughout. In further agreement with the global correlation, K27 methylation differed more dramatically between MEFs and ES/iPS cells than K4 methylation. Based on the previous observation that developmental genes are the most important target group of PcG-mediated K27 methylation in murine ES cells (Boyer et al., 2006), it was decided to test if these loci are enriched within signature genes. Indeed, gene ontology analysis revealed that developmental genes are the most significantly enriched gene group in the E class of signature genes (p=8×e⁻¹⁰). These findings suggested that changes in K27 methylation are more significant for the reprogramming from MEFs into iPS cells than changes in K4 methylation and suggest an important role for PcG proteins in reprogramming.

To test if the correlation of the iPS and ES cell histone methylation patterns faithfully captures changes in the transcriptional status of the iPS cells, expression analysis was performed on ES cells, 2D4 iPS cells, and MEFs at the whole genome level using Agilent microarrays. ES and iPS cells showed a very high correlation in expression patterns at the global level as determined by Pearson correlation (FIGS. 11A and 11B). Genes with a more than two-fold difference in expression between ES cells and MEFs were almost identically expressed between ES and iPS cells (data not shown). Therefore, these data indicate that iPS cells, as expected from the epigenetic data, are transcriptionally highly comparable to ES cells. The levels of a randomly chosen subset of 13 signature genes were confirmed by real time RT-PCR (FIG. 11C). All tested genes were expressed at similar levels in iPS cells and ES cells. The differences in expression of signature genes between ES, iPS cells, and MEFs correlated well with the observed differences in the histone methylation patterns (data not shown), suggesting that K4 and K27 methylation are important determinants of the expression state of those genes. Taken together, these data demonstrate that nuclear reprogramming by four transcription factors can induce global transcriptional and epigenetic resetting of the fibroblast genome.

### Example 8

### MEF and TTF-Derived iPS Cells Differentiate into Numerous Cell Types Including Germ Cells

It was reasoned that the faithful epigenetic reprogramming of iPS cells will result in a developmental potential that is comparable to that of ES cells. Injection of GFP marked MEF-derived 2D4 iPS cells into diploid blastocysts gave rise to three newborn chimeras with obvious GFP fluorescence (FIG. 8A, Table 2). Tissue sections from a newborn pup showed broad and clonal contribution of iPS cells to the cartilage, glandular structures, liver, heart, and lungs (data not shown). FACS analysis of hematopoietic cells derived from a newborn pup revealed that between 18-28% of splenic B cells and macrophages as well as thymic CD4+ and CD8+ T cells were derived from iPS cells (FIG. 8B). Moreover, it was possible to isolate iPS cell-derived tail fibroblasts and neurosphere cultures from this chimeric pup, which showed similar growth rates and cytokine dependence compared with host-derived fibroblasts and neurospheres (data not shown). One chimera that developed into adulthood showed coat color chimerism, indicating differentiation of iPS cells into functional melanocytes (FIG. 8C).

It was next asked if, in addition to MEF-derived iPS cells, female iPS cells could also support development. Blastocyst injection of two different iPS clones that had been selected based on the re-expression of a Xi^{GFP} transgene gave rise to one postnatal animal per line (see Table 2). The chimeric animals appeared healthy and grew normally into adult mice. These results indicate that iPS cells derived from TFTs, like iPS cells derived from fetal fibroblasts, give rise to normal appearing postnatal chimeras.

Germ line transmission is considered one of the most stringent tests for the pluripotency of cells. To assess whether Xi^{GFP}/X TTF-derived iPS cells can contribute to the germ line, 16 oocytes were isolated from one super-ovulated iPS chimera of which 4 were brightly GFP positive, indicating contribution of IFS cells to the female germ line (data not shown). Treatment of these oocytes with strontium chloride and cytochalasin B resulted in successful parthenogenetic activation and subsequent cleavage to the blastocyst stage, thus demonstrating functionality of oocytes (data not shown).

Directed differentiation of ES cells into mature cell types has clear therapeutic potential. To determine whether iPS cells give rise to mature cells in vitro, EBs were generated that were explanted in culture to induce hematopoietic cell fates. Indeed, cell types were detected expressing markers of immature and mature blood cells, thus underscoring the potential use of iPS cells in regenerative medicine (FIG. 12).

The generation of pluripotent cells directly from fibroblast cultures has represented a major advance towards understanding the mechanisms that govern nuclear reprogramming (Takahashi and Yamanaka, 2006). Here, the first evidence is provided that faithful epigenetic resetting of the genome accompanies transcription factor-induced reprogramming. iPS cells were recovered that were remarkably similar to ES cells in their epigenome. For example, female iPS cells showed proper demethylation at the promoters of key pluripotency genes, they reactivated a somatically silenced X chromosome that underwent random X inactivation upon differentiation, and they had a global histone methylation pattern that was almost identical to that of ES cells. iPS cells also revealed other ES-like qualities including growth factor responsiveness, the ability to act as reprogramming donors in cell fusion, as well as the ability to undergo ES-like differentiation both in vitro and in vivo, contributing to high-grade postnatal chimeras including one germ line chimera.

The finding that transgenic Oct4 expression is not required for the maintenance of iPS cells indicates that the endogenous gene expression program has been sufficiently reactivated to ensure maintenance of pluripotency. This indicates that exogenous expression of Oct4 and possibly also that of Sox2, c-Myc and Klf4 may only be necessary during the initial steps of reprogramming to trigger transcriptional and epigenetic changes that lead to pluripotency. In support of this notion, retroviral expression of the four factors was high in infected donor fibroblasts and silenced in iPS cells. Thus, it is feasible to transiently supply somatic cells with the four factors, generating stably reprogrammed cells that do not contain retroviral or transgenic elements, which may result in insertional mutagenesis or gene expression artifacts, respectively.

Surprisingly, Nanog-selected iPS cells were phenotypically and molecularly different from the previously reported Fbx 15-selected iPS cells. Nanog is essential for embryonic development and is required for the maintenance of pluripotency by suppressing differentiation into primitive endoderm (Chambers, 1., Colby, D., Robertson, M., Nichols, J., Lee, S., Tweedie, S., and Smith, A. (2003) Cell 113, 643-655.; Mitsui, K., Tokuzawa, Y., Itoh, H., Segawa, K., Murakami, M., Takahashi, K., Maruyama, M., Maeda, M., and Yamanaka, S. (2003) Cell 113, 631-642). Fbx15, in contrast, is not essential for pluripotency or development despite its exclusive expression in ES cells (Tokuzawa, Y., Kaiho, E., Maruyama, M., Takahashi, K., Mitsui, K., Maeda, M., Niwa, H., and Yamanaka, S. (2003) Mol Cell Biol 23, 2699-2708). While not wishing to be bound by theory, there are several possible explanations for the qualitative differences between Fbx15 selected iPS cells and the iPS cells described herein. One possibility is that Nanog selection gives rise to a different pluripotent cell type with greater developmental potential compared with Fbx15 selection. In agreement, most Fbx15-selected iPS cells did not express Nanog (Takahashi and Yamanaka, 2006), which may explain why they inappropriately differentiated in the absence of MEFs and failed to give rise to full-term chimeras. In further support of this notion is the observation that not all Oct4 expressing cells are also positive for Nanog in normal ES cell cultures, suggesting heterogeneity within the ES cell population (Hatano et al., 2005). Interestingly, inner mass cells of the blastocyst, from which ES cells are derived, show a similarly heterogeneous expression pattern for Oct4 and Nanog Chazaud, C., Yamanaka, Y., Pawson, T., and Rossant, J. (2006) Dev Cell 10, 615-624.).

Again not wishing to be bound by theory, an alternative explanation for the effect of Nanog selection on the quality of resultant iPS cells could be that Nanog protein itself plays a critical role in faithful epigenetic reprogramming. In agreement with this idea, cell fusion experiments between ES cells and somatic cells have shown to result in 200-fold more colonies when Nanog is overexpressed in ES cells (Silva, J., Chambers, I., Pollard, S., and Smith, A. (2006) Nature 441, 997-1001). Although Nanog is not required for inducing pluripotency in somatic cells, it is informative to assess whether its overexpression during the reprogramming process enhances the efficiency of obtaining iPS cells, and if it affects the developmental potency of iPS cells.

Again not wishing to be bound by theory, another possibility for the observed differences between the previously reported iPS cells and the iPS cells described herein may be the timing of selection. It was not possible to derive iPS cells from Nanog-GFP-puro MEFs when selection was applied three days after infection, which is in contrast to the findings by Yamanaka and colleagues, who were able to select for Fbx15 expression at this time. Hence, selection was started one week after infection, or isolated iPS cells solely based on ES cell morphology or the reactivation of a silenced X-linked GFP transgene, followed by retrospective verification of pluripotency using the Oct4-Neo allele. All iPS cells derived without initial drug selection appeared better than the previously reported Fbx15-selected iPS cells in terms of chimeric contribution and ES cell-like epigenetic features. It is hypothesized that reprogramming is a gradual process that takes several days or weeks and depends on a cascade of genes that need to be reactivated. In this scenario, Nanog reactivation might occur later during nuclear reprogramming than Fbx15 reactivation. Thus, early selection for Fbx15 may expand a cell population that has not completed nuclear reprogramming, consequently eliminating potentially better reprogrammed cells that would appear later during the reprogramming process; late selection for Nanog may capture a stage at which reprogramming is more complete. One way to probe this hypothesis would be to test whether late selection for Fbx 15 expression generates iPS cells that are more similar to ES cells. The observation that morphological selection of ES-like colonies instead of drug selection can be sufficient for obtaining iPS cells has important implications for direct reprogramming in humans, as introducing reporter transgenes into human cells is technically challenging and may cause insertional mutagenesis.

Direct reprogramming of cells to pluripotency has clear therapeutic implications, and it has therefore been crucial to ascertain whether iPS cells exist in the same epigenetic state as ES cells. These data indicate that abnormal epigenetic reprogramming should not compromise the therapeutic utility of directly reprogrammed cells.

### Example 9

### Human iPS Cells can be Generated in the Absence of Selection

Patient-specific fibroblasts or keratinocytes were infected with the four (OCT4, SOX2, CMYC, KLF4) or five (4+NANOG) reprogramming factors that were expressed by a tetracycline-inducible lentiviral system. The viruses were co-infected with a lentivirus expressing the reverse tetracycline transactivator (rtTA). The cells were passaged onto feeder cells and induced with doxycycline; the cells were kept in fibroblast media for the first 3 days, then switched to human ES cell conditions. Small colony-like structures became visible within 4 days; by 30 days, colonies with human ES cell morphology were present with a distinct hES-like cobblestone appearance (data not shown). Colonies with a non-hES cell morphology were also present but did not interfere with the generation of the hES-like colonies.

The hES-like colonies were picked, expanded, and characterized. Like human ES cells, they were pluripotent (generated teratomas), expressed key pluripotency genes, and showed proper re-setting of epigenetic modifications. In addition, they had also silenced the lentiviral transgenes.

All references, including any patents or patent applications cited in this specification, as well as the figures and table, are hereby incorporated by reference. No admission is made that any reference constitutes prior art. The discussion of the references states what their authors assert, and the applicants reserve the right to challenge the accuracy and pertinence of the cited documents. It will be clearly understood that, although a number of prior art publications are referred to herein, this reference does not constitute an admission that any of these documents form part of the common general knowledge in the art, in the United States of America or in any other country.

TABLE 1
Table S1. Summary of iPS cell lines obtained

| | | Onset of | | % SSEA- | Developmental Potential | |
|---|---|---|---|---|---|---|
| Parent cell | iPS cell | selection | Morphology | 1+ | Teratoma | Chimera |
| Nanog | 1A2 | 7 days | Both ES like cells and small round | 63.2** | Pluripotent (teratoma | Live-born |
| GFPiresPuro | | | cells; sorted and subcloned to | | after 4 weeks) | |
| MEFs | | | obtain ES-like population* | | | |
| (female) | 1B3 | 7 days | Both ES like cells and small round | 21.2 | Tumor consisting of | ND |
| | | | cells | | hematopoietic cells | |
| | 1D4 | 3 weeks | Identical to ES cells | 65.0 | Pluripotent (teratoma | ND |
| | | | | | after 3 weeks) | |
| | 2B3 | 7 days | Both ES like cells and small round | 20.7 | Small teratoma | ND |
| | | | cells | | obtained after 8 weeks | |
| | 2D4 | 3 weeks | Identical to ES cells | 79.0 | Pluripotent (teratoma | Live-born |
| | | | | | after 3 weeks) | |
| Oct4-Neo | 1 | After colony | Identical to ES cells | ND | ND | ND |
| Xa/Xi^{GFP} TTFs | | picking | | | | |
| (female) | 2 | After colony | Identical to ES cells | ND | ND | Live-born |
| | | picking | | | | |
| | 3 | After colony | Identical to ES cells | ND | ND | Live-born |
| | | picking | | | | |
| | 4 | After colony | Identical to ES cells | ND | ND | ND |
| | | picking | | | | |
| Oct4-Neo, | 1 | After colony | Identical to ES cells | ND | ND | ND |
| Oct4-inducible | | picking | | | | |
| TTFs (female) | 2 | After colony | Identical to ES cells | ND | Pluripotent (teratoma | ND |
| | | picking | | | after 3 weeks) | |

*Cells were triple sorted for GFP, SSEA-1, and CD9, then subcloned to obtain a homogenous stable ES-like population;
**Analysis performed after triple sorting;
ND = not determined

TABLE 2
Table S2. Efficiencies of term development and estimated
degree of chimerism in IPS cell-derived mice.

| | | | | % chimerism |
|---|---|---|---|---|
| | | | # chimeric | (based on |
| | | | mice | GFP signal |
| IPS cell lines | # blastocysts | # pups born | (% pups | or coat |
| (donor cell) | injected | (% blastocysts) | born) | color) |
| 1A2-10 | 15 | 4 (27%) | 1 (25%) | 30% |
| (Nanog-GiP | | | | |
| MEF) | | | | |
| 2D4-7 (Nanog- | 35 | 6 (17%) | 3 (50%) | 30-70% |
| GiP MEF) | | | | |
| OT-2 (Oct4- | 12 | 6 (50%) | 1 (17%) | 10% |
| Neo XGFP | | | | |
| TTF) | | | | |
| OT-3 (Oct4- | 15 | 3 (20%) | 1 (33%) | 30% |
| Neo XGFP | | | | |
| TTF) | | | | |

TABLE 3
Table S3: Primer sequences for RT-PCR analyses and Antibodies

| Gene | FIG. | Primer sequence Forward | Primer sequence Reverse |
|---|---|---|---|
| Cripto | 1C | ATGGACGCAACTGTGAACATGATGTTCGCA | CTTTGAGGTCCTGGTCCATCACGTGACCAT |
| ERas | 1C | ACTGCCCCTCATCAGACTGCTACT | CACTGCCTTGTACTCGGGTAGCTG |
| Nanog | 1C | CAGGTGTTTGAGGGTAGCTC | CGGTTCATCATGGTACAGTC |
| Nat1 | 1C | ATTCTTCGTTGTCAAGCCGCCAAAGTGGAG | AGTTGTTTGCTGCGGAGTTGTCATCTCGTC |
| Sox2 | 1C | TAGAGCTAGACTCCGGGCGATGA | TTGCCTTAAACAAGACCACGAAA |
| Oct3/4 | 1C, 3D | GCTATCTACTGTGTGTCCCAGTC | AGAGAAGGATGTGGTTCGAG |
| Xite | 5D | ATTCAGGCGTGGTAGACATC | GTGGGGCGCAAAATGTCTAG |
| region 5* | | | |
| Xite | 5D | TCTGAGTACATAAGGGCCAC | GTAGACTTTCGTAAGTCCCC |
| region 6* | | | |
| Xite | 5D | TTTCCGGAGGAAGCCTGAAC | CTCCTGATCCTCTTATCTGG |
| region 7* | | | |
| Rrm2* | 5D | AAGCGACTCACCCTGGCTGAC | GACTATGCCATCACTCGCTGC |
| Rassf1 | Supp 6D | GGACTACAATGGCCAGATCAA | GGAAGGCACTGAAACAGGAC |
| Trh | Supp 6D | AGGAAAGACCTCCAGCGTGT | TCTCTTCGGCTTCAACGTCT |
| Grh13 | Supp 6D | TCCAGCACATTGAAGAGGTG | GCGAGGAGAAGTCTGTGCTC |
| Dppa4 | Supp 6D | GGAGGGAAAACCACAAGACA | CTGTCTTCAACCTGGCGTCT |
| Arid5b | Supp 6D | CAACAGTGGGCTCAACTTCA | GGGGGTAACTGAGCACAATC |
| Aspn | Supp 6D | AGGACACGTTCAAGGGAATG | ACTGTCACCCCTTCAAATGC |
| Nuak1 | Supp 6D | CGTTCACCGAGATCTCAAGC | GAACGTCTGGAGGAACTTGC |
| Trib2 | Supp 6D | ATCTGCACAGCGGAGAGG | CGTGATTTGGTTGATGTTGC |
| Rest | Supp 6D | CCTGCAGCAAGTGCAACTAC | GCTTGAGTAAGGACAAAGTTCACA |
| Fgf7 | Supp 6D | CCATGAACAAGGAAGGGAAA | TCCGCTGTGTGTCCATTTAG |
| Vgll4 | Supp 6D | CAGTGACACAGGCAGGTCAG | GGGACAGTGAGAGAGGTTGC |
| Fgd4 | Supp 6D | ATGGGATTGGATACGTTGGA | CCGGCTGACATAAGCTCTTT |
| Hoxd10 | Supp 6D | CTGAGGTTTCCGTGTCCAGT | TTCTGCCACTCTTTGCAGTG |
| Gapdh | Supp 6D | TTCACCACCATGGAGAAGGC | CCCTTTTGGCTCCACCCT |
| pMX-Sox2 | 1E | CCCATGGTGGTGGTACGGGAATTC | TCTCGGTCTCGGACAAAAGT |
| pMX-Klf4 | 1E | CCCATGGTGGTGGTACGGGAATTC | CGTTGAACTCCTCGGTCT |
| pMX-Oct4 | 1E | CCCATGGTGGTGGTACGGGAATTC | AGTTGCTTTCCACTCGTGCT |
| pMX-cMYC | 1E | CTCCTGGCAAAAGGTCAGAG | TCGGTTGTTGCTGATCTGTC |
| Beta | 1E, 3D | TGTTACCAACTGGGACGACA | TCTCAGCTGTGGTGGTGAAG |
| Actin | | | |
| Inducible | 3D | ATCCACGCTGTTTGACCTC | CGAAGTCTGAAGCCAGGTGT |
| Oct4 | | | |
| allele | | | |

| Antibodies | Company |
|---|---|
| cMyc | Santa Cruz, sc-789 |
| Klf4 | Sante Cruz, sc-20691 |
| Nanog | Abcam, AB21603 |
| Oct3/4 | Santa Cruz sc-5279 |
| Oct3/4 | Santa Cruz sc-8628 for |
| Sox2 | immunostaining |
| β-Actin | Chemicon, AB5603 |
| γ-Tubulin | Sigma, A5441 |
| Ezh2 | T6557 |
| PolII | BD612667 |
| H3me3K27 | Upstate 05-623 |
| H3me3K27 | Upstate 05-851 for immunostaining |
| H4me1K20 | Upstate 07-449 |
| Gata4 | Abcam 9051 |
| Rabbit IgG | Santa Cruz sc9053 |
| H3me3K4 | Upstate 12-370 |
| PE-conjugated anti-mouse CD41 | Abcam 8580 |
| APC-conjugated anti-mouse c-kit | Pharmingen MWReg30 |
| PECy7-conjugated anti-mouse | eBiosciences 2B8 |
| CD45 | eBiosciences 30-F11 |
| biotinylated CD4 | eBiosciences L3T4 |
| biotinylated CD8a | eBiosciences 53-6.7 |
| biotinylated CD19 | eBiosciences 1D3 |
| biotinylated CD11b | eBiosciences M1/70 |
| CD16/32 | eBiosciences 93 |

*Otawa Y. and Lee. J. T. Xite, X-inactivation intergenic transcption elements that regulate the probability of choice. Mol. Cell. 2003 11:731-743.

1. A method of selecting induced pluripotent stem cells, the method comprising:
a) re-programming a differentiated primary cell to a pluripotent phenotype, wherein the differentiated primary cell does not express Nanog mRNA when measured by RT-PCR;
b) culturing the cell re-programmed in step (a) in the absence of a selection agent after re-programming;
c) microscopically observing the culture of step (b), and isolating a clone of cells in the culture which have become smooth and rounded in appearance; and
d) testing cells of the clone for the expression of a stem cell marker; wherein the detection of stem cell marker expression is indicative that the cells are induced pluripotent stem cells.

2. The method of claim 1 wherein said re-programming comprises one of: introducing nucleic acid sequences encoding the transcription factors Oct4, Sox2, c-Myc and Klf4 to said differentiated somatic cell, the sequences operably linked to regulatory elements for the expression of the factors; introducing one or more protein factors that re-program the cell's differentiation state; and contacting said cell with a small molecule that induces a re-programming of the cell's differentiated state.

3. The method of claim 1 further comprising the step of introducing cells of a said clone that express a stem cell marker into nude mice and performing histology on a tumor arising from the cells, wherein the growth of a tumor comprising cells from all three germ layers further indicates that the cells are pluripotent stem cells.

4. The method of claim 1 wherein the step of culturing further comprises passaging said cells.

5. The method of claim 1 wherein said differentiated somatic cell has a morphology distinctly different from that of an ES cell.

6. The method of claim 1 wherein the differentiated primary cell is a fibroblast, and wherein said fibroblast is flattened and irregularly shaped prior to said re-programming.

7. The method of claim 1 wherein the stem cell marker is selected from the group consisting of SSEA1, CD9, Nanog, Fbx15, Ecat1, Esg1, Eras, Gdf3, Fgf4, Cripto, Dax1, Zpf296, Slc2a3, Rex1, Utf1, and Oct4.

8. The method of claim 1, further comprising, when the differentiated primary cell is from a female individual, the step of testing cells of the clone for the reactivation of an inactive X chromosome.

9. The method of claim 2 wherein said nucleic acid sequences are comprised in a viral vector or a plasmid.

10. The method of claim 9 wherein said viral vector is a retroviral vector, a lentiviral vector or an adenoviral vector.

11. The method of claim 1 further comprising the step of testing cells of said clone for the expression of exogenous Oct4, Sox2, c-Myc and/or Klf4.

12. The method of claim 1, wherein said cell comprises a human cell.

13. A method of selecting induced pluripotent stem cells, the method comprising:
a) providing a female cell that is heterozygous for a selectable marker on the X chromosome, wherein the selectable marker is mutant on the active X chromosome and wild-type on the inactive X chromosome, and wherein the cell does not express Nanog mRNA when measured by RT-PCR;
b) re-programming said cell to a pluripotent phenotype;
c) culturing the cell with a selection agent, wherein the reactivation of the inactive X chromosome permits the expression of wild-type selectable marker and permits cell survival in the presence of the selection agent, whereby surviving cells are induced pluripotent stem cells.

14. The method of claim 13, further comprising the step of testing a cell surviving in the presence of the selection agent for the expression of a stem cell marker.

15. The method of claim 14, wherein the stem cell marker is selected from the group consisting of SSEA1, CD9, Nanog, Fbx15, Ecat1, Esg1, Eras, Gdf3, Fgf4, Cripto, Dax1, Zpf296, Slc2a3, Rex1, Utf1, and Oct4.

16. The method of claim 13, wherein said re-programming comprises one of: introducing nucleic acid sequences encoding the transcription factors Oct4, Sox2, c-Myc and Klf4 to said differentiated somatic cell, the sequences operably linked to regulatory elements for the expression of the factors; introducing one or more protein factors that re-program the cell's differentiation state; and contacting said cell with a small molecule that induces a re-programming of the cell's differentiated state.

17. The method of claim 13, further comprising the step of introducing cells that survive in the presence of the selection agent into nude mice and performing histology on a tumor arising from the cells, wherein the growth of a tumor comprising cells from all three germ layers further indicates that the cells are pluripotent stem cells.

18. The method of claim 13, wherein the cell is a cell of a cell line.

19. The method of claim 13, wherein the cell is heterozygous for a mutant Hprt gene on the X chromosome.

20. The method of claim 19 wherein the cell carries a wild-type Hprt gene on the X chromosome that is inactive before the introduction of the nucleic acids and a mutant, non-functional Hprt gene on the X chromosome that is active before said re-programming.

21. The method of claim 13, wherein the cell is resistant to 6-thioguanine before said re-programming.

22. The method of claim 13, wherein the selection agent comprises HAT medium.

23. The method of claim 13, wherein said cell comprises a human cell.

24. A method of selecting induced pluripotent stem cells, the method comprising:
a) providing a female cell which carries an X-chromosome-linked reporter gene that is subject to silencing by X inactivation, and wherein said female cell does not express Nanog mRNA when measured by RT-PCR;
b) re-programming said cell to a pluripotent phenotype;
c) culturing the cell after said re-programming; and
d) isolating a clone of cells from the culture which expresses the X-chromosome-linked reporter; wherein the expression of the reporter is indicative that the clone comprises induced pluripotent stem cells.

25. The method of claim 24, further comprising the step of testing cells of the clone for the expression of a stem cell marker.

26. The method of claim 25, wherein the stem cell marker is selected from the group consisting of SSEA1, CD9, Nanog, Fbx15, Ecat1, Esg1, Eras, Gdf3, Fgf4, Cripto, Dax1, Zpf296, Slc2a3, Rex1, Utf1, and Oct4.

27. The method of claim 24, further comprising the step of introducing cells that express the reporter into nude mice and performing histology on a tumor arising from the cells, wherein the growth of a tumor comprising cells from all three germ layers further indicates that the cells are pluripotent stem cells.

28. The method of claim 24, wherein said cell comprises a human cell.
